(19) United States
(12) Patent Application Publication
(10) Pub. No.: US 2020/0222418 A1
(43) Pub. Date: Jul. 16, 2020

(54) USE OF PHENOTHIAZINE DERIVATIVE IN THE TREATMENT OF INFECTION CAUSED BY BACTERIA CARRYING TYPE IV PILI

(71) Applicants: Centre National de la Recherche Scientifique (CNRS), Paris (FR); Institute National de la Sante et de la Recherche Medicale (INSERM), Paris (FR); Universite Paris Descartes, Paris (FR)

(72) Inventors: Sandrine Bourdoulous, Gagny (FR); Kévin Denis, La Varenne Saint-Hilaire (FR); Loïc Le Guennec, Paris (FR)

(73) Assignees: Centre National de la Recherche Scientifique (CNRS), Paris (FR); Institut National de la Sante et de la Recherche Medicale (INSERM), Paris (FR); Universite Paris Descartes, Paris (FR)

(21) Appl. No.: 16/628,687

(22) PCT Filed: Jul. 6, 2018
(86) PCT No.: PCT/EP2018/068393

§ 371 Date: Jan. 7, 2020

(30) Foreign Application Priority Data
Jul. 7, 2017 (EP) 17305893.4

(51) Int. Cl. A61K 31/5415; A61K 9/00; A61K 45/06; A61P 31/04

(52) U.S. Cl. CPC A61K 31/5415; A61P 31/04; A61K 45/06; A61K 9/0019

(57) ABSTRACT

The present invention provides a phenothiazine derivative of formula (I) for use in preventing and/or treating infection caused by bacteria carrying Type IV pili, such as N. meningitidis, and more specifically for use in preventing and/or treating meningitis. The present invention further relates to a composition for the use in preventing and/or treating infection caused by bacteria carrying Type IV pili, such as purpura fulminans and meningitis, comprising a phenothiazine derivative of formula (I) and an antibiotic selected from the group consisting of beta-lactams and aminoglycosides, and/or dexamethasone.

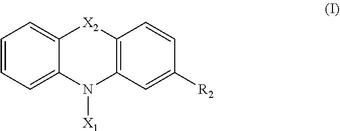

### BACKGROUND

The present invention is directed to phenothiazine derivatives for use in preventing and/or treating infection of bacteria carrying Type IV pili including Gram negative bacteria, such as e.g. Neisseria meningitidis (meningococcus), Klebsiella sp., notably Klebsiella pneumoniae, Haemophilus sp., notably Haemophilus influenzae, Escherichia coli sp., notably Escherichia coli K1, Enteropathogenic Escherichia coli (EPEC) and Enterotoxigenic E. coli (ETEC), Pseudomonas sp., notably Pseudomonas aeruginosa, Vibrio cholerae, Acinetobacter baumanii, Yersinia pseudotuberculosis, Legionella pneumophila, Neisseria gonorrhoeae, Salmonella enterica and Moraxella catarrhalis; and including Gram positive bacteria such as e.g. Staphylococcus sp., notably Staphylococcus aureus, Clostridium perfringens, Clostridium difficile, Streptococcus sp., notably Streptococcus pneumoniae and Streptococcus agalactiae, and Listeria monocytogenes.

Meningococcal type IV pili are long filamentous structures that extend from the bacterial cell surface. They mediate different functions including DNA uptake, twitching motility, bacterial aggregation, adhesion to endothelial cells and signaling events that eventually lead to bacterial translocation through endothelia (Doulet et al. 2006; Coureuil et al. 2009 et 2010; Dupin et al. 2012). These structures are heteromultimeric assemblies of pilin subunits that form helical fibers (Craig et al. 2008). The major pilin subunit, called PilE in Neisseria spp., is assembled in a polymeric helical fiber and constitutes the essential fiber scaffold. Neisseria type IV pilins have been grouped in two classes (class I and class II). Other less abundant (‘minor’) pilins, such as PilV, PilX and ComP, structurally resemble PilE and participate in various pilus-related functions such as adhesion, bacterial aggregation and DNA uptake, respectively (Helaine et al. 2005; Mikaty et al. 2009; Brown et al. 2010). Both PilE and PilV have been shown to be involved in adhesion to host cells (Coureuil et al. 2010; Stephens et al. 1985; Nassif et al. 1994; Virji et al. 1993; Winther-Larsen et al. 2001).

The endothelium lining blood and lymphatic vessels is a key barrier separating circulating body fluids from host tissues and is a major target for several pathogenic bacteria (Lemichez et al. 2010). Indeed, a crucial step in the pathogenesis of invasive infections by piliated bacteria is the adhesion of bloodborne bacteria to both peripheral and brain endothelia, a process referred to as vascular colonization (Melican et al. 2012).

For example, N. meningitidis, also referred to as meningococcus, is an obligate human Gram-negative bacterium that normally resides in the nasopharyngeal mucosa without affecting the host, a phenomenon known as carriage (Yazdankhah et al. 2004). Pathology is initiated when meningococci gain access to the bloodstream, multiply in the blood and disseminate into various tissues, leading to complete blockage of the blood vessels, vascular dysfunction, local thrombotic responses, which in the most serious cases, leads to the breach of the endothelial barrier and the formation of necrotic lesions (purpura fulminans).

After interaction with brain microvessels, meningococci promote vessel occlusion, cross the blood-brain barrier and reach the meninges and the CSF where they multiply, causing meningitis (Brandtzaeg et al. 2012).

The pathogenic capacity of type IV pili capsulated meningococci has been shown to rely on the tight interaction between human endothelial cells and bacterial type IV pili (Virji et al. 2009; Lemichez et al. 2010). Furthermore, in vivo studies showed that bacterial mutants with altered type IV pili function did not trigger inflammation or lead to vascular damage showing that local type IV pili-mediated adhesion of meningococci to the vascular wall, as opposed to circulating bacteria, determines vascular dysfunction in meningococcemia (Join-Lambert et al, 2013; Melican et al. 2013).

Invasive infections by piliated bacteria can also lead to various diseases, such as pharyngitis (e.g. Pneumococcus, N. meningitidis, Haemophilus), gastrointestinal and pulmonary infections (e.g. E coli K1, ETEC, EPEC, C. perfringens, C. difficile, Pseudomonas aeruginosa, Vibrio cholerae, Yersinia pseudotuberculosis, Moraxella, Streptococcus 8), sepsis (e.g. Pneumococcus, N. meningitidis, Haemophilus, E. coli K1, Pseudomonas aeruginosa), meningitis (e.g. Pneumococcus, N. Meningitis, Haemophilus, E. coli K1, Streptococcus pneumonia, Listeria monocytogenes) and cutaneous infections (e.g. Pseudomonas aeruginosa).

Further diseases are known to be related to bacteria carrying Type IV pili, in particular joint arthritis (e.g. N. meningitidis, Neisseria gonorrhoeae, Moraxella catarrhalis); and purpura fulminans, coronaritis, endocarditis, pericarditis, mycotic aortic aneurysm, pleurisy, conjunctivitis, endophthalmitis or brain abscesses (e.g. N. meningitidis).

Early treatment of infections caused by piliated bacteria, such as meningococcal infections, relies on intravenous administration of antibiotics: beta-lactams, such as cephalosporins (i.e. cefotaxime, ceftriaxone), penicillin (benzyolpenicillin), gentamicin or chloramphenicol in patients who are allergic to penicillin. This early antibiotic therapy improves survival of patients and increases the chances of limiting the harmful effects of infection.

However, despite antimicrobial therapy, systemic infections by piliated bacteria, such as meningococcal infections, remain a major cause of mortality and severe neurological sequelae (Stephens et al. 2007; Virji et al. 2009; Brandtzaeg et al. 2012).

Moreover, more than 45 percent of patients being treated for sepsis with antibiotic therapy develop acute kidney injury, which is a serious and common health complication. Indeed, high doses antibiotic therapy, required to cross the blood-brain barrier, often aggravate acute renal failure due to septic shock, requiring the need for extra-renal blood purification, with all the potential complications arising therefrom, gram that may worsen the course of pre-existing disseminated intravascular coagulation process. Further, the antibiotic therapy efficiency is also challenged, as antibiotics only kill bacteria but does not disperse the bacterial aggregates formed at the endothelial cell surface. As a consequence, dead bacterial aggregates still promote signalling events leading to vascular damages, i.e. alteration of the endothelial cell junctions, degradation of the basement membrane. Moreover, antibiotic therapy by killing bacteria, releases pro-inflammatory and toxic bacterial compounds, which promote inflammation suspected as the cause of adverse effects associated with the initiation of treatment. Hence, by dispersing the bacterial aggregate formed in the bloodstream this inflammation is reduced. This led to the advice to start with a low dose of the antibiotic.

The frequently observed worsening of clinical symptoms after initiation of antibiotic treatment during sepsis or meningitis led to the concept of immunosuppression by corticosteroids and other agents concomitant or prior to the first antibiotic dose.

A therapeutic approach in meningitis and sepsis, which avoids immunosuppression, is to influence the liberation of bacterial pro-inflammatory cell wall products using bactericidal antibiotics or other compounds, which would delay or even circumvent bacterial lysis (Neu, 2012).

Over the past two decades, numerous studies have been undertaken to discover novel therapeutic strategies adjunct to antibiotics. Since both inflammation and coagulation play a pivotal role in the pathogenesis of sepsis, clinical trials were carried out with anti-inflammatory or anti-thrombotic treatments for controlling a deleterious systemic inflammatory host response, or to try to reduce the disorders of hemostasis and disseminated intravascular coagulation processes observed during high vascular colonization by bacteria carrying Type IV pili, such as N. meningitidis. However, these trials were not effective for N. meningitidis infection, with unchanged mortality rate.

Consequently, there is a need to discover new molecules or compositions to prevent and/or treat efficiently infection caused by bacteria carrying Type IV pili, such as N. meningitidis infection, which does not present the inconvenient of the prior art treatments.

In this context, the inventors unexpectedly found that a treatment with an antipsychotic selected from the group of phenothiazine derivative, such as trifluoperazine, was able to block all the functions carried by the type IV pili (bacterial competence, twitching motility, aggregation and adhesion to inert surface or host endothelial cells) in different bacterial pathogens.

More specifically, there is a need to discover new molecules or compositions to prevent and/or treat efficiently meningitis caused by bacteria carrying Type IV pili, which do not present the inconvenients of the prior art treatments.

Therefore, by blocking the functions carried by the type IV pili, phenothiazine derivatives have been shown to:
Induce the loss of Type IV pili (acts on pilus dynamics);
Prevent bacterial aggregation and adhesion to human endothelial cells;
Dissociate bacterial aggregates, and consequently reduce vascular colonization by bacteria carrying Type IV pili, such as N. meningitidis and prevent an overwhelming inflammatory response, therefore conferring an advantage over antibiotics treatment;
Induce the loss of Type IV pili (acts on pilus dynamics);
Reduce large scale systemic dysregulation by stopping endothelial cells from receiving intracellular signals;
Reduce the endothelial cell cytoskeleton and basal membrane remodeling following bacterial adhesion;
Protect vascular cell junctions integrity; and
Prevent subsequent vascular damages.

### DETAILED DESCRIPTION OF THE INVENTION

The present invention relates to a phenothiazine derivative or a pharmaceutical salt thereof for use in preventing and/or treating infections caused by bacteria carrying Type IV pili including Gram negative bacteria, such as e.g. Neisseria meningitidis, Klebsiella sp., notably Klebsiella pneumoniae, Haemophilus sp., notably Haemophilus influenzae, Escherichia coli sp., notably Escherichia coli K1, Enteropathogenic Escherichia coli (EPEC) and Enterotoxigenic E. coli (ETEC), Pseudomonas sp., notably Pseudomonas aeruginosa, Vibrio cholerae, Acinetobacter baumanii, Yersinia pseudotuberculosis, Legionella pneumophila, Neisseria gonorrhoeae, Salmonella enterica and Moraxella catarrhalis; and including Gram positive bacteria such as e.g. Staphylococcus sp., notably Staphylococcus aureus, Clostridium perfringens, Clostridium difficile, Streptococcus sp., notably Streptococcus pneumoniae and Streptococcus agalactiae, and Listeria monocytogenes.

The present invention also relates to a phenothiazine derivative or a pharmaceutical salt thereof for use in treating and/or preventing meningitis caused by bacteria carrying Type IV pili, wherein said phenothiazine derivative is a compound of formula (I):

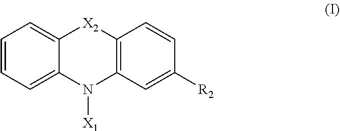

X₁ is a C₁-C₆ alkyl substituted by R₁;

wherein R₁ is YR₃R₄, wherein
Y is C or N,
If Y is N then R₃ and R₄ are independently of each other H, (C₁-C₆)alkyl,
or R3 and R4 form together with Y, a ring selected from an heteroaryl or an heterocyclyl group, said ring being optionally substituted by one or more groups selected from:
   (C₁-C₆)alkyl optionally substituted by OH or O(C═O)(C1-C10)alkyl; or
   an amide group, and
If Y is C then R₃ and R₄ form together with Y, a ring selected from an heteroaryl or an heterocyclyl group, said ring being optionally substituted by (C₁-C₆)alkyl groups optionally substituted by OH;

R₂ is H, an halogen (preferably Cl or F), CF₃, a (C₁-C₆)alkoxy, S(O)(C₁-C₆)alkyl, SO₂ (C₁-C₆) alkyl, SO₃H, CN, a (C₁-C₆)alkyl, a (C₁-C₆)thioalkoxy, NO₂

X₂ is S or SO₂.

The present invention also relates to a pharmaceutical composition for use in preventing and/or treating infections caused by bacteria carrying Type IV pili, said pharmaceutical composition comprising a phenothiazine derivative or an acceptable pharmaceutical salt thereof, and preferentially an antibiotic selected from the group of beta-lactams and aminoglycosides, and/or dexamethasone.

More specifically, the present invention relates to a pharmaceutical composition for use in preventing and/or treating meningitis caused by bacteria carrying Type IV pili, said pharmaceutical composition comprising a phenothiazine derivative or an acceptable pharmaceutical salt thereof, and an antibiotic selected from the group consisting of beta-lactams and aminoglycosides, and/or dexamethasone.

The present invention further relates to a kit comprising a phenothiazine derivative or a pharmaceutical salt thereof, and/or at least one antibiotic.

More specifically, said antibiotic is selected from the group consisting of beta-lactams and aminoglycosides, and/or dexamethasone.

Another object of the present invention is a method of treating patients diagnosed or at risk of developing infection caused by piliated bacteria, comprising the step of administering a therapeutically effective amount of a phenothiazine derivative or a pharmaceutical salt thereof according to the invention. In particular, said infection caused by piliated bacteria is meningitis.

The present invention is also directed to a method of preventing infection caused by piliated bacteria, comprising the step of administering to patient a therapeutically effective amount of a phenothiazine derivative or a pharmaceutical salt thereof according to the invention. In particular, said infection caused by piliated bacteria is meningitis.

Indeed, the inventors found that the use of a phenothiazine derivative or a pharmaceutical salt thereof dissociates bacterial aggregates formed at the surface of infected endothelial cells, such as N. meningitidis aggregates. Even more interestingly the compounds of the invention are capable of dispersing bacterial aggregates, even when said bacteria were pre-treated with antibiotics. Therefore, a phenothiazine derivative or a pharmaceutical salt thereof can be used to treat or prevent vascular dysfunctions and further endothelial colonization by circulating piliated bacteria, such as N. meningitidis.

Indeed, the inventors found that the use of a phenothiazine derivative or a pharmaceutical salt thereof rapidly induces the loss of type IV pili from piliated bacteria, e.g. Gram negative bacteria, such as e.g. Neisseria meningitidis, Klebsiella sp., notably Klebsiella pneumoniae, Haemophilus sp., notably Haemophilus influenzae, Escherichia coli sp., notably Escherichia coli K1, Enteropathogenic Escherichia coli (EPEC) and Enterotoxigenic E. coli (ETEC), Pseudomonas sp., notably Pseudomonas aeruginosa, Vibrio cholerae, Acinetobacter baumanii, Yersinia pseudotuberculosis, Legionella pneumophila, Neisseria gonorrhoeae, Salmonella enterica and Moraxella catarrhalis; and Gram positive bacteria such as e.g. Staphylococcus sp., notably Staphylococcus aureus, Clostridium perfringens, Clostridium difficile, Streptococcus sp., notably Streptococcus pneumoniae and Streptococcus agalactiae, and Listeria monocytogenes. As a result, phenothiazine derivatives prevent adhesion of piliated bacteria to endothelial cells, dissociate bacterial aggregates that are already formed at the surface of infected endothelial cells and prevent the activation of subsequent signalling pathways leading to vascular insults. An advantage of the dissociation of bacterial aggregates is the reduction of inflammation due to dispersion in the bloodstream of bacterial pro-inflammatory compounds.

This effect differs from antibiotic treatment, since antibiotics, although they kill bacteria, do not induce complete bacterial dispersion at the endothelial cell surface. Antibiotic-treated bacteria still promote signalling events leading to vascular damages, i.e. alteration of the endothelial cell junctions, degradation of the basement membrane, whereas phenothiazine derivatives, used alone or in combination with antibiotics, drastically reduced endothelial cell colonization and subsequent endothelial dysfunctions.

Further, the inventors showed that phenothiazine derivatives or a pharmaceutical salt thereof inhibit host cell signalling events promoted by piliated bacteria, such as N. meningitidis. For example, Ezrin recruitment and actin polymerization at the bacterial adhesion sites were prevented by the drug. Likewise, treatment with a phenothiazine derivative or a pharmaceutical salt thereof inhibits the loss of VE-cadherin at the endothelial cell junctions, which is caused by the bacteria.

Finally, the inventors found that the use of a phenothiazine derivative or a pharmaceutical salt thereof inhibits the recruitment of PECAM-1 (Platelet and Endothelial Cell Adhesion Molecule 1, CD31) from the intercellular junctions of the endothelial cells to the bacterial adhesion sites. PECAM-1 is a major component of endothelial cell intercellular junctions, where it contributes importantly to barrier function and control of vascular permeability (Ferrero et al., 1995; Graesser et al., 2002) and confers protection against endotoxic shock (Carrithers et al., 2005; Maas et al., 2005). Likewise, treatment with a phenothiazine derivative or a pharmaceutical salt thereof further contributes to exert a vasculoprotective effect on infected endothelial cells by maintaining PECAM-1 localization at the endothelial cell junctions.

Therefore, a phenothiazine derivative or a pharmaceutical salt thereof can be used to treat or prevent vascular dysfunctions and further endothelial colonization by circulating piliated bacteria, such as N. meningitidis.

In a preferred embodiment, the phenothiazine derivative or a pharmaceutical salt thereof can be used to treat or prevent infections caused by Gram negative bacteria, such as e.g. Neisseria meningitidis, Klebsiella sp., notably Klebsiella pneumoniae, Haemophilus sp., notably Haemophilus influenzae, Escherichia coli sp., notably Escherichia coli K1, Enteropathogenic Escherichia coli (EPEC) and Enterotoxigenic E. coli (ETEC), Pseudomonas sp., notably Pseudomonas aeruginosa, Vibrio cholerae, Acinetobacter baumanii, Yersinia pseudotuberculosis, Legionella pneumophila, Neisseria gonorrhoeae, Salmonella enterica and Moraxella catarrhalis; or caused by Gram positive bacteria such as e.g. Staphylococcus sp., notably Staphylococcus aureus, Clostridium perfringens, Clostridium difficile, Streptococcus sp., notably Streptococcus pneumoniae and Streptococcus agalactiae, and Listeria monocytogenes.

In one embodiment, the phenothiazine derivative or a pharmaceutical salt thereof can be used to treat or prevent:
pharyngitis caused by pneumococcus, N. meningitidis, haemophilus;
gastrointestinal and pulmonary infections caused by E. coli K1, ETEC, EPEC, C. perfringens, C. difficile, Pseudomonas aeruginosa, Vibrio cholerae, Yersinia, Moraxella, Streptococcus agalactiae;
sepsis caused by pneumococcus, N. meningitidis, Haemophilus, Streptococcus agalactiae E. coli K1, Pseudomonas aeruginosa;
meningitis caused by pneumococcus, N. meningitidis, Haemophilus sp., Streptococcus agalactiae, E. coli K1, Streptococcus pneumonia, Listeria monocytogenes;
cutaneous infections caused by Pseudomonas aeruginosa;
endocarditis caused by Streptococcus agalactiae;
joint arthritis caused by N. meningitidis, Neisseria gonorrhoeae, Moraxella catarrhalis; and
purpura fulminans, coronaritis, endocarditis, pericarditis, mycotic aortic aneurysm, pleurisy, conjunctivistis, endophthalmitis or brain abscesses caused by N. meningitidis.

In a preferred embodiment, the infection of piliated bacteria according to the invention is a pharyngitis, a gastrointestinal infection, a pulmonary infection, a sepsis, a meningitis, a cutaneous infection, endocarditis, joint arthritis, purpura fulminans, coronaritis, endocarditis, pericarditis, mycotic aortic aneurysm, pleurisy, conjunctivistis, endophthalmitis or brain abscesses, preferentially purpura fulminans, meningitis or pharyngitis, more preferentially is purpura fulminans or a meningitis, and in the most preferred embodiment is meningitis.

By “bacteria carrying Type IV pili or piliated bacteria” it is meant any bacterium exposing at its surface Type IV pili, i.e. Gram negative bacteria, such as e.g. Neisseria meningitidis, Klebsiella sp., notably Klebsiella pneumoniae, Haemophilus sp., notably Haemophilus influenzae, Escherichia coli sp., notably Escherichia coli K1, Enteropathogenic Escherichia coli (EPEC) and Enterotoxigenic E. coli (ETEC), Pseudomonas sp., notably Pseudomonas aeruginosa, Vibrio cholerae, Acinetobacter baumanii, Yersinia pseudotuberculosis, Legionella pneumophila, Neisseria gonorrhoeae, Salmonella enterica and Moraxella catarrhalis; and Gram positive bacteria such as e.g. Staphylococcus sp., notably Staphylococcus aureus, Clostridium perfringens, Clostridium difficile, Streptococcus sp., notably Streptococcus pneumoniae and Streptococcus agalactiae, and Listeria monocytogenes.

The term “preventing” as used herein refers to avoiding the onset of a condition such as infection caused by bacteria carrying Type IV pili, as used herein or its accompanying syndromes. It will be understood that prevention refers to avoiding the onset of said condition within a certain time window in the future. Said time window shall preferably start upon administration of a phenothiazine derivative in the sense of the invention and lasts for at least 1 week, at least 1 month. It will be understood that prevention may not be successful for 100% of the subjects to be treated. The term “preventing”, however, requires that the prevention is successful for a statistically significant portion of the subjects (e.g. a cohort in a cohort study). Whether a portion is statistically significant can be determined without further ado by the person skilled in the art using various well-known statistic evaluation tools discussed also elsewhere herein in detail.

The terms “treat,” “treated,” or “treating” as used herein refers to both therapeutic treatment and prophylactic or preventative measures, wherein the object is to prevent or slow down an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

A phenothiazine derivative according to the invention is a compound of formula (I):

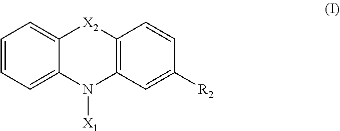

X₁ is a C₁-C₆ alkyl substituted by R₁;

wherein R₁ is YR₃R₄, wherein
Y is C or N,
If Y is N then R₃ and R₄ are independently of each other H, (C₁-C₆)alkyl,
or R₃ and R₄ form together with Y, a ring selected from an heteroaryl or an heterocyclyl group, said ring being optionally substituted by one or more groups selected from:
   (C₁-C₆)alkyl optionally substituted by OH or O(C═O)(C1-C10)alkyl; or
   an amide group, and
If Y is C then R₃ and R₄ form together with Y, a ring selected from an heteroaryl or an heterocyclyl group, said ring being optionally substituted by (C₁-C₆)alkyl groups optionally substituted by OH;

R₂ is H, an halogen (preferably Cl or F), CF₃, a (C₁-C₆)alkoxy, S(O)(C₁-C₆)alkyl, SO₂(C₁-C₆) alkyl, SO₃H, CN, a (C₁-C₆)alkyl, a (C₁-C₆)thioalkoxy, NO₂;

X₂ is S or SO₂.

The term “(C₁-C₁₀)alkyl”, as used in the present invention, refers to a straight or branched saturated hydrocarbon chain containing from 1 to 10 carbon atoms including, but not limited to, methyl, ethyl, n-propyl, iso-propyl, n-butyl, iso-butyl, sec-butyl, t-butyl, n-pentyl, n-hexyl, decanyl and the like. Preferably it is a methyl group.

The term “(C₁-C₆)alkoxy”, as used in the present invention, refers to a (C₁-C₆)alkyl group as defined above bound to the molecule via an oxygen atom, including, but not limited to, methoxy, ethoxy, n-propoxy, iso-propoxy, n-butoxy, iso-butoxy, sec-butoxy, t-butoxy, n-pentoxy, n-hexoxy, and the like. Preferably it is a methoxy group.

The term “(C₁-C₆)thioalkoxy”, as used in the present invention, refers to a (C₁-C₆)alkyl group as defined above bound to the molecule via a sulfur atom, including, but not limited to, thiomethoxy, thioethoxy, n-thiopropoxy, iso-thiopropoxy, n-thiobutoxy, iso-thiobutoxy, sec-thiobutoxy, t-thiobutoxy, n-thiopentoxy, n-thiohexoxy, and the like. Preferably it is a thiomethoxy group.

The term “heteroaryl” as used herein alone or as part of another group denotes optionally substituted aromatic groups having at least one heteroatom in at least one ring, and preferably 5 or 6 atoms in each ring. The heteroaryl group preferably has 1 to 3 heteroatoms preferably selected from O, N and S in the ring, and may be bonded to the remainder of the molecule through a carbon or heteroatom. Exemplary heteroaryl groups include imidazolyl, pyridyl, pyrrolyl, pyrimidinyl, pyrazinyl, tetrazolyl, triazolyl and triazinyl.

The term “heterocyclyl”, as used in the present invention, refers to a hydrocarbon monocyclic or bicyclic (fused) ring having 3 to 10 ring atoms, containing at least one heteroatom, preferably 1 or 2 heteratoms, in the ring. The heteroatom is preferably selected from O, N or S, and the S atom may be mono or dioxidized, i.e. the sulphur atom may be S, S(O) or SO₂. Heterocyclyl groups include, but are not limited to piperidinyl, piperazinyl, morpholinyl, pyrrolidinyl and aziridinyl.

Phenothiazine derivatives according to the invention can be classified into three groups that differ in respect of the substituent present on nitrogen: aliphatic compounds bear acyclic groups, piperidines bear piperidine-derived groups, and piperazines bear piperazine-derived substituents. Preferably, a “phenothiazine derivative” as used herein refers to a compound selected from the group consisting of:
aliphatic compounds comprising chlorpromazine, oxomémazine, promazine, trifluopromazine and levomepromazine;
piperidines comprising mesoridazine and thioridazine; and
piperazines comprising prochlorperazine, fluphenazine, trifluoperazine and perphenazine.

In a preferred embodiment, a phenothiazine derivative according to the invention is selected from the piperidine and piperazine groups. Advantageously the said phenothiazine derivative according to the invention is promazine, mesoridazine, thioridazine, trifluoperazine, prochlorperazine, fluphenazine, or perphenazine, more preferably thioridazine, mesoridazine, trifluoperazine, prochlorperazine, or perphenazine, even more preferably thioridazine, mesoridazine or trifluoperazine. These compounds and their synthesis pathways are well known from the skilled person.

Another object of the present invention relates to a method of treating patients diagnosed with, or suspected to have, infection caused by bacteria carrying Type IV pili comprising the step of administering a therapeutically effective amount of promazine, mesoridazine, thioridazine, trifluoperazine, prochlorperazine, fluphenazine and perphenazine, preferably thioridazine, mesoridazine, trifluoperazine, prochlorperazine, fluphenazine and perphenazine, more preferably thioridazine, mesoridazine or trifluoperazine.

In a preferred embodiment, said infection caused by bacteria carrying Type IV pili is meningitis.

The present invention also relates to a method of preventing infection caused by bacteria carrying Type IV pili, comprising the step of administering to a patient a therapeutically effective amount of promazine, mesoridazine, thioridazine, trifluoperazine, prochlorperazine, fluphenazine or perphenazine, preferably thioridazine, mesoridazine, trifluoperazine, prochlorperazine, fluphenazine or perphenazine, more preferably thioridazine, mesoridazine or trifluoperazine.

In a preferred embodiment, said infection caused by bacteria carrying Type IV pili is meningitis.

As used herein, the term “purpura” refers to any accumulation of blood in the skin due to vascular extravasation, irrespective of size or cause, and to medical conditions commonly referred to as “petechiae” (pinpoint spots), “ecchymoses” (larger macular (flat) patches) and “purpura” (larger spots). Purpura results from the extravasation of blood from the vasculature into the skin or mucous membranes. Purpura, in general, is hemorrhage of blood out of the vascular spaces and into the surrounding tissues of the skin or mucous membranes. This hemorrhage results in a collection of blood in the dermis of the skin that is visible initially as a dark purple/red discoloration that changes color as it breaks down and is resorbed.

By “infectious purpura” it is meant herein any purpuric lesion caused by a Gram-negative bacterium (N. meningitidis, Haemophilus influenzae, Pseudomonas aeruginosa, and others) or Gram-positive organisms (Staphylococcus aureus, group B streptococci, Streptococcus pneumonia, and others), preferentially a Gram-negative bacterium.

By “purpura fulminans” it is meant a severe form of infectious purpura. Purpura fulminans is a rapidly progressive syndrome of intravascular thrombosis and hemorrhagic infarction of the skin. It includes large purpuric skin lesions, fever, hypotension and disseminated intravascular coagulation (DIC). The term purpura fulminans also encompassed herein the corresponding adult pathology “symmetrical peripheral gangrene” or “ischemic skin lesions” (Lerolle et al. 2013).

The term “meningitis” as used herein refers to an acute inflammation of the protective membranes, covering the brain and spinal cord, known collectively as “meninges”. The most common symptoms are fever, headache, and neck stiffness. However, meningitis can also be life-threatening because of the proximity of the inflammation to the brain and spinal cord.

Meningitis may be caused by a variety of factors, such as e.g. infection with viruses, bacteria, etc.

Bacterial meningitis notably is particularly dangerous. Several strains of bacteria can cause accurate bacterial meningitis, notably Streptococcus pneumonia, Neisseria meningitidis, Haemophilus influenzae and Listeria monocytogenes.

In specific embodiment of the invention, bacteria carrying Type IV pili are selected from the group consisting of Streptococcus pneumoniae, Neisseria meningitidis, Haemophilus sp., notably Haemophilus influenzae, and Listeria monocytogenes.

More specifically, the bacterium is Neisseria meningitidis.

The phenothiazine derivatives of the invention are useful for preventing an infection caused by bacteria carrying Type IV pili.

The phenothiazine derivatives of the invention are particularly useful for preventing N. meningitidis infection, to evolve into a more severe form of the condition, such as e.g. meningitis or purpura fulminans.

Thus, the present invention is also directed to a method of preventing infection caused by bacteria carrying Type IV pili, said method comprising the step of administering to patient a therapeutically effective amount of a phenothiazine derivative or a pharmaceutical salt thereof according to the invention.

In a preferred embodiment, said infection caused by bacteria carrying Type IV pili is meningitis.

In a preferred embodiment, the method of preventing infection caused by bacteria carrying Type IV pili is a method of preventing N. meningitidis infection, said method comprising the step of administering to patient presenting purpuric lesions a therapeutically effective amount of a phenothiazine derivative or a pharmaceutical salt thereof according to the invention.

In a preferred embodiment, said infection caused by N. meningitidis is meningitis.

In particular, as explained above, N. meningitidis infections are characterized by vascular damages. Such damages result from the formation of large bacterial aggregates at the apical surface of the endothelium. The present inventors have shown that the phenothiazine derivatives or pharmaceutical salt thereof of the invention can disperse such aggregates, demonstrating that they can be used for the prevention and/or the treatment of vascular damages associated with N. meningitidis infection.

Another advantage of the present invention is the reduction of inflammation due to the dispersion of the bacterial aggregates caused by bacteria carrying Type IV pili, which disperse in the bloodstream bacterial pro-inflammatory and toxic compounds.

The inventors have also shown that the phenothiazine derivative or pharmaceutical salt thereof of the invention can inhibit the recruitment at the bacterial adhesion sites of endothelial cell junction proteins, such as PECAM-1, a protein known to play a crucial role in the stabilization of cell-cell contacts at the lateral junctions of endothelial cells and in the maintenance of the vascular permeability (Wong et al., 2004; Park et al., 2010; Fernandez-Martin et al., 2012)

In one embodiment, the phenothiazine derivative or pharmaceutical salt thereof for use in preventing and/or treating N. meningitidis infection according to the invention prevents vascular damages. In a preferred embodiment, said N. meningitidis infection is meningitis.

The expression “vascular damages” refers herein to any vascular disorder associated with diminished functionality of the vessels walls. Such disorders include, e.g. vascular congestion and dilation, endothelial necrosis, increase in endothelial permeability, alteration of markers of endothelial integrity (VE-cadherin, PECAM-1/CD31) and of the protein C pathway receptors (endothelial protein C receptor, thrombomodulin). Important vascular damages can also lead to circulatory collapse.

By “circulatory collapse” it is meant a failure of the circulatory system fails to maintain the supply of oxygen and other nutrients to the tissues and to remove the carbon dioxide and other metabolites from them. Circulatory collapse can result from capillary leak syndrome (also known as systemic capillary leak syndrome, SCLS, or Clarkson's disease) or vascular damages, intravascular volume depletion, vasodilation, and myocardial dysfunction. If the body's compensatory mechanisms are overwhelmed, hypotension occurs, resulting in tissue hypoxia and acidosis, which further impairs myocardial function. In addition, myocarditis, pericarditis, or direct bacterial invasion of the heart can also induce myocardial dysfunction. Thus, circulatory collapse can refer to a “cardiac circulatory collapse” when it affects the vessels of the heart (aorta), or to a “peripheral circulatory collapse” when it affects outlying arteries and veins in the body, that can result in gangrene, organ failure or other serious complications.

The compound of the invention is particularly advantageous since it could be used to prevent and/or treat vascular damages and/or circulatory collapse. Preferentially, the phenothiazine derivative or a pharmaceutical salt thereof for use in treating infection caused by bacteria carrying Type IV pili, such as N. meningitidis infection, according to the invention also prevents circulatory collapse. According to a preferred embodiment, said infection is caused by Neisseria meningitidis.

N. meningitidis is a Gram-negative bacterium and member of the bacterial family Neisseriaceae. Meningococcal virulence is related to both capsule expression, expression of other surface structures, and underlying genotype. Capsule of N. meningitidis helps with transmission, as it protects the N. meningitidis from desiccation, phagocytic killing, opsonisation and complement-mediated bactericidal killing. There are 13 serogroups of N. meningitidis based on different capsular polysaccharide structures, but only 6 serogroups (A, B, C, W-135, X, and Y) are responsible for most infections. In a preferred embodiment, the phenothiazine derivative or a pharmaceutical salt thereof according to the invention are preferentially used to treat infections caused by N. meningitidis presenting the serogroups A, B, C, W-135, X and Y.

The effectiveness of the phenothiazine derivative of the invention in preventing and/or treating infection caused by bacteria carrying Type IV pili, can be improved by administering phenothiazine derivatives of the invention serially or in combination with another agent that is effective for those purposes. Usual treatment of infection caused by bacteria carrying Type IV pili, such as N. meningitidis infection, or such as meningitis, involves the administration of antibiotics. For example, beta-lactams such as e.g., cephalosporins have been shown to be particularly efficient in killing infecting bacterial cells, such as e.g. N. meningitidis. This early antibiotic therapy improves patient survival. However, once vascular colonization is established, antibiotics are not efficient in preventing subsequent vascular insults/purpuric lesions. Moreover, antibiotic therapy in high doses, required to cross the blood-brain barrier, may also aggravate acute renal failure due to septic shock.

Unlike antibiotics, the phenothiazine derivatives of the invention act not by killing infectious bacteria, but by disaggregating the clusters of bacteria present at the surface of blood vessels. The combination of phenothiazine derivatives and antibiotics thus displays an unexpected synergistic effect in preventing vascular lesions and inflammation. This finding suggests that the use of phenothiazine derivatives in combination with antibiotics allow reducing dose regimen of antibiotics (cephalosporins) and their side effects, such as renal failure.

Antibiotics used in treatment of infection caused by bacteria carrying Type IV pili, such as N. meningitidis infections or meningitis, comprise in particular beta-lactams including cephalosporins, but also aminoglycosides, notably gentamicin, and other antibiotics such as chloramphenicol.

In the context of the invention, the term “ß-lactam” refers to any antibiotic containing a ß-lactam ring in its molecular structure. The ß-lactams of the invention thus comprise penicillin derivatives as well as cephalosporins, monobactams, carbapenems and ß-lactamase inhibitors. In particular, the ß-lactam of the invention can be benzylpenicillin (penicillin G), phenoxymethylpenicillin (penicillin V), ampicillin (penicillin A), benzathine benzylpenicillin, methicillin, dicloxacillin, flucloxacillin, co-amoxiclav (amoxicillin+clavulanic acid), piperacillin, ticarcillin, azlocillin, carbenicillin, cephalexin, cefalotin, cefazolin, cefaclor, cefuroxime, cefamandole, cefotetan, cloxacillin, cefadroxil, cefixime, cefoxitin, ceftriaxone, cefotaxime, ceftazidime, cefepime, cefpirome, imipenem, imipenem in combination with cilastatin, cefixime in combination with imipenem, meropenem, mecillinam, ertapenem, aztreonam, clavulanic acid, tazobactam or sulbactam. Preferably, beta-lactams according to the present invention are cephalosporins or benzylpenicillin (penicillin G).

By “cephalosporin” it is meant any third-generation cephalosporin selected from cefcapene, cefdinir, cefditoren, cefetamet, cefixime, cefmenoxime, cefoperazone, cefotaxime cefpiramide, cefpodoxime, cefsulodin, ceftibuten, ceftizoxime, ceftriaxone, latamoxef (or moxalactam) and flomoxef.

Common treatment of infection caused by bacteria carrying Type IV pili, such as N. meningitidis infection or meningitis, may also rely on the administration of an aminoglycoside such as gentamicin, or of chloramphenicol. Such antibiotics are in particular useful in patients who are allergic to penicillin. By “aminoglycoside”, it is herein referred to a medicinal and bacteriologic category of traditional Gram-negative antibacterial therapeutic agents that inhibit protein synthesis and contain as a portion of the molecule an amino-modified glycoside. Aminoglycosides include such antibiotics as streptomycin, kanamycin, tobramycin, gentamicin and neomycin.

Preferably the aminoglycoside of the invention is gentamicin. “Gentamicin” as used herein refers to an antibiotic obtained from the bacteria Micromonospora purpurea and which has the structure defined by the formula (3R,4R,5R)-2-{[(1S,2S,3R,4S,6R)-4,6-diamino-3-{[(2R,3R,6S)-3-amino-6-[(1R)-1-(methylamino)ethyl]oxan-2-yl]oxy}-2-hydroxycyclohexyl]oxy}-5-methyl-4-(methylamino)oxane-3,5-diol. Gentamicin is commonly used to treat many types of bacterial infections including bone infections, endocarditis, pelvic inflammatory disease, meningitis, pneumonia, urinary tract infections and sepsis among others.

Alternatively, the antibiotic which is administered along with the phenothiazine derivatives of the invention or pharmaceutical salt thereof is a third-generation cephalosporin (C3G) antibiotic such as cefotaxime or ceftazidime.

Accordingly, the invention relates to a combination of a phenothiazine derivative or pharmaceutical salt thereof and an antibiotic for use in treating and/or preventing infection caused by bacteria carrying Type IV pili.

More specifically, the invention relates to a combination of a phenothiazine derivative or pharmaceutical salt thereof and an antibiotic for use in treating and/or preventing meningitis caused by bacteria carrying Type IV pili.

The invention also relates to a method of treatment or prevention of infection caused by bacteria carrying Type IV pili, wherein said method comprises the step of administering a combination of a phenothiazine derivative or pharmaceutical salt thereof and an antibiotic to a patient in need thereof. Preferably, said antibiotic is selected in the group consisting of beta-lactams, aminoglycosides, notably gentamicin and a third-generation cephalosporin (C3G).

In particular, the infection caused by bacteria carrying Type IV pili is meningitis, and more particularly is meningitis caused by bacteria selected from the group consisting of: Streptococcus pneumonia, Neisseria meningitidis, Haemophilus sp., notably Haemophilus influenza, and Listeria monocytogenes. Preferably the involved bacterium is Neisseria meningitidis.

On suspicion of bacterial meningitis, it is recommended to give dexamethasone five minutes before antibiotic therapy to reduce the risk of inflammation. By “dexamethasone”, it is herein referred to a corticosteroid of formula 8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one. Dexamethasone is commonly used to treat many different inflammatory conditions such as allergic disorders, skin conditions, ulcerative colitis, arthritis, lupus, psoriasis, or breathing disorders. In particular, dexamethasone is known for its property to reduce neurologic sequelae associated with bacterial meningitis.

The invention thus also relates to a combination of a phenothiazine derivative or pharmaceutical salt thereof and dexamethasone for use in treating and/or preventing infection caused by bacteria carrying Type IV pili, such as N. meningitidis infection or meningitis.

The invention also relates to a method of treating or preventing infection caused by bacteria carrying Type IV pili, such as N. meningitidis infection or meningitis, wherein said method comprises the step of administering a combination of a phenothiazine derivative or pharmaceutical salt thereof and dexamethasone to a patient in need thereof. Preferably, said combination also comprises an antibiotic. More preferably, said antibiotic is selected in the group consisting of beta-lactams, aminoglycosides, notably gentamicin and a third-generation cephalosporin.

In another aspect, the present invention relates to a pharmaceutical composition comprising a phenothiazine derivative or pharmaceutical salt thereof, and at least one pharmaceutically acceptable excipient.

The pharmaceutical composition of the invention may contain, in addition to the excipient and phenothiazine derivative or pharmaceutical salt thereof, various diluents, fillers, salts, buffers, stabilizers, solubilizers, and other materials well known in the art.

Said pharmaceutical composition may further comprise an antibiotic as described above and/or dexamethasone.

Preferentially, the present invention thus relates to a pharmaceutical composition for use in preventing and/or treating infection caused by bacteria carrying Type IV pili, said pharmaceutical composition comprising a phenothiazine derivative or an acceptable pharmaceutical salt thereof, and an antibiotic selected in the group consisting of beta-lactams and aminoglycosides, and/or dexamethasone.

In a preferred embodiment, the invention relates to a pharmaceutical composition for the use in preventing and/or treating meningitis caused by bacteria carrying Type IV pili, comprising a phenothiazine derivative and an antibiotic selected in the group consisting of beta-lactams and aminoglycosides, and/or dexamethasone.

In a more specific embodiment, said meningitis is caused by bacteria carrying Type IV pili selected from the group consisting of: Streptococcus pneumonia, Neisseria meningitidis, Haemophilus sp., notably Haemophilus influenza, and Listeria monocytogenes, and is preferably caused by the bacterium species Neisseria meningitidis.

As used herein, “pharmaceutically acceptable excipient” includes any and all solvents, buffers, salt solutions, dispersion media, coatings, antibacterial and antifungal agents, isotonic and absorption delaying agents, and the like that are physiologically compatible. The type of excipient can be selected based upon the intended route of administration. In various embodiments, the excipient is suitable for intravenous, intraperitoneal, subcutaneous, intramuscular, topical, transdermal or oral administration. Pharmaceutically acceptable excipients include sterile aqueous solutions or dispersions and sterile powders for the extemporaneous preparation of sterile injectable solutions or dispersion. The use of media and agents for pharmaceutically active substances is well known in the art. As detailed here below, additional active compounds can also be incorporated into the compositions, such as other antibiotics; in particular, the additional antibiotic is selected in the group consisting of beta-lactams, aminoglycosides, notably gentamicin and a third-generation cephalosporin antibiotic. A typical pharmaceutical composition for intravenous infusion could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17th ed., Mack Publishing Company, Easton, Pa. (1985), and the 18^{th} and 19^{th} editions thereof, which are incorporated herein by reference.

The phenothiazine derivative or pharmaceutical salt thereof in the composition preferably is formulated in an effective amount. An “effective amount” refers to an amount effective, at dosages and for periods of time necessary, to achieve the desired result, such as decrease, blockage, or reversal of infection caused by bacteria carrying Type IV pili, such as N. meningitidis infection. A “therapeutically effective amount” means an amount sufficient to influence the therapeutic course of a particular disease state. The activity contemplated by the present methods includes both medical therapeutic and/or prophylactic treatment, as appropriate. A therapeutically effective amount is also one in which any toxic or detrimental effects of the agent are outweighed by the therapeutically beneficial effects. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages will normally fall within the range of from 0.01 mg to 1000 mg a day, administered in only one dose once a day or in several doses along the day, for example twice a day. The daily administered dose is advantageously comprised between 5 mg and 500 mg, and more advantageously between 10 mg and 200 mg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

For therapeutic applications, the phenothiazine derivative or pharmaceutical salt thereof is administered to a human, in a pharmaceutically acceptable dosage form such as those discussed above, including those that may be administered to a human intravenously as a bolus or by continuous infusion over a period of time, by intramuscular, intraperitoneal, intracerebrospinal, subcutaneous, intraarticular, intrasynovial, intrathecal, oral, topical, or inhalation routes, preferentially intravascular or intramuscular.

The phenothiazine derivative or pharmaceutical salt thereof can be administered in unit forms for administration, mixed with conventional pharmaceutical carriers, to a human. Suitable unit forms for administration comprise the forms for oral administration, such as tablets, gelatin capsules, powders, granules and oral solutions or suspensions, the forms for sublingual and buccal administration, the forms for subcutaneous, intramuscular, intravenous, intranasal or intraocular administration and the forms for rectal administration, preferentially intravascular or intramuscular.

The administration routes, dosing schedules and optimal galenic forms can be determined according to the criteria generally taken into account when establishing a treatment suited to a patient such as, for example, the patient's age or body weight, the seriousness of his general state, his tolerance for the treatment and the side effects experienced. Dosage regimens may be adjusted to provide the optimum response. For example, a single bolus may be administered, several divided doses may be administered over time, or the dose may be proportionally reduced or increased.

For parenteral, intranasal or intraocular administration, aqueous suspensions, isotonic saline solutions or sterile and injectable solutions which contain pharmacologically compatible dispersing agents and/or wetting agents are used. The active principle may also be formulated in the form of microcapsules, optionally with one or more carrier additives.

When a solid composition is prepared in the form of tablets, the main active ingredient is mixed with a pharmaceutical vehicle such as gelatin, starch, lactose, magnesium stearate, talc, gum arabic and the like. The tablets may be coated with sucrose or with other suitable materials, or they may be treated in such a way that they have a prolonged or delayed activity and they continuously release a predetermined amount of active principle. A preparation in gelatin capsules is obtained by mixing the active ingredient with a diluent and pouring the mixture obtained into soft or hard gelatin capsules. A preparation in the form of syrup or elixir may contain the active ingredient together with a sweetener, an antiseptic, or also a taste enhancer or a suitable coloring agent. The water-dispersible powders or granules may contain the active ingredient mixed with dispersing agents or wetting agents, or suspending agents, and with flavor correctors or sweeteners.

For rectal administration, suppositories are used which are prepared with binders which melt at rectal temperature, for example cocoa butter or polyethylene glycols. For intramuscular injection a medical device, such as a syringe, is used to deliver a medication deep into the muscles, to allow the medication to be absorbed into the bloodstream quickly.

As explained above, one or more other agents, e.g. an antibiotic and/or dexamethasone, may be present in the composition being administered or may be administered separately. In one aspect of the invention, the administration is performed with the other active principle, e.g. an antibiotic and/or dexamethasone, either simultaneously, separately or sequentially over time. When the administration is performed simultaneously, the two active principles may be combined in a single pharmaceutical composition, comprising the two compositions, such as a tablet or a gel capsule. On the other hand, the two active principles may, whether or not they are administered simultaneously, be present in separate pharmaceutical compositions. To this end, the combination may be in the form of a kit comprising, on the one hand, the phenothiazine derivative or pharmaceutical salt thereof, as described above and, on the other hand, the second active principle, e.g. an antibiotic and/or dexamethasone, the phenothiazine derivative or pharmaceutical salt thereof as described above and the second active principle being in separate compartments and being intended to be administered simultaneously, separately, or sequentially over time.

The present invention also includes kits, e.g., comprising a phenothiazine derivative or pharmaceutical salt thereof and/or at least one antibiotic, or a combination comprising said phenothiazine derivative or pharmaceutical salt thereof and/or at least one antibiotic, and instructions for the use of said phenothiazine derivative or pharmaceutical salt thereof and/or at least one antibiotic, or of a combination comprising said phenothiazine derivative or pharmaceutical salt thereof and/or at least one antibiotic for treating infection caused by bacteria carrying Type IV pili, such as N. meningitidis infection or meningitis. The instructions may include directions for using the phenothiazine derivative or pharmaceutical salt thereof and/or at least one antibiotic, or a combination comprising said phenothiazine derivative or pharmaceutical salt thereof and/or at least one antibiotic, in vivo. Typically, the kit will have a compartment containing the phenothiazine derivative or pharmaceutical salt thereof and/or at least one antibiotic, or a combination comprising said phenothiazine derivative or pharmaceutical salt thereof and/or at least one antibiotic. The phenothiazine derivative or pharmaceutical salt thereof and/or at least one antibiotic, or a combination comprising said phenothiazine derivative or pharmaceutical salt thereof and/or at least one antibiotic may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the phenothiazine derivative or pharmaceutical salt thereof and/or at least one antibiotic, or a combination comprising said phenothiazine derivative or pharmaceutical salt thereof and/or at least one antibiotic prior to administering to a patient, and tools that aid in administering the phenothiazine derivative or pharmaceutical salt thereof and/or at least one antibiotic, or a combination comprising said phenothiazine derivative or pharmaceutical salt thereof and/or at least one antibiotic, to a patient.

In a preferred embodiment, the kit according to the invention comprises a phenothiazine derivative and an antibiotic selected in the group consisting of beta-lactams and aminoglycosides, and/or dexamethasone. Preferentially, said phenothiazine derivative are selected from thioridazine, mesoridazine, trifluoperazine, prochlorperazine, fluphenazine and perphenazine, preferably from thioridazine, mesoridazine and trifluoperazine and said antibiotic is a beta-lactam, preferably a cephalosporin of third generation.

The examples that follow are merely exemplary of the scope of this invention and content of this disclosure. One skilled in the art can devise and construct numerous modifications to the examples listed below without departing from the scope of this invention.

### LEGENDS OF THE FIGURES

FIG. 1: Antimicrobial activity of Trifluoperazine on N. meningitidis in vitro.

N. meningitidis Nm2C4.3 strain grown in liquid culture at 10⁷ CFU/ml were treated with increasing concentrations of Trifluoperazine (10 to 50 μM) or Gentamicin (150 μg/ml) for 15 minutes (A) Bactericidal activity was determined by the count of colony forming units on GCB agar plates 24 h after treatment. (B) Images of the non-treated bacteria or bacteria treated with 30 μM TFP grown for 24 h on GCB agar plates.

FIG. 2: Trifluoperazine inhibits meningococcal aggregation.

N. meningitidis 2C4.3 was grown in liquid culture at 10⁷ CFU/ml for 2 h to form bacterial aggregates and were treated with increasing concentrations of Trifluoperazine (10 to 40 μM), Gentamicin (150 μg/ml) or cefotaxim (20 μg/ml) for 20 minutes and bacterial aggregates were immediately visualized using a phase-contrast microscope. (A) Representatives images of the bacterial aggregates observed in phase-contrast microscope in non-treated or treated conditions. (B) Quantification of the bacterial aggregation was performed using Image J software.

FIG. 3: Trifluoperazine rapidly inhibits the aggregation of meningococcal wild type strain whereas it does not affect the aggregation of a PilT derivative mutant.

N. meningitidis 2C4.3 wild type strain and the isogenic derivative mutant PilT (□PiIT) were grown in liquid culture at 10⁷ CFU/ml for 2 h to form bacterial aggregates and then treated with 50 μM Trifluoperazine. Time lapse phase-contrast video microscopy was performed to visualize the effect on bacterial aggregates over time. Images were taken at the indicated time points of the video.

FIG. 4: The effect of Trifluoperazine on meningococcal aggregation is reversible/transient.

N. meningitidis 2C4.3 was grown in liquid culture at 10⁷ CFU/ml for 2 h to form bacterial aggregates and were treated with 30 μM Trifluoperazine for 30 minutes to induce bacterial dispersion or PBS as a control. The medium was replaced to remove the Trifluoperazine and the reformation of bacterial aggregates were visualized overtime using a phase-contrast microscope.

FIG. 5: Trifluoperazine induces the loss of bacterial type IV pili

N. meningitidis 2C4.3 wild type strain and the isogenic derivative mutant PilT (□PiIT) were grown in liquid culture at 10⁷ CFU/ml for 2 h to form bacterial aggregates and then treated with 50 μM Trifluoperazine for 15 minutes before analysis by transmission electron microscopy. Arrows point at bundles of type IV pili expressed at the bacterial surface.

FIG. 6: Chemical Structure of phenothiazine derivatives used in this study.

Aliphatic compounds (Promazine, Chlorpromazine, Triflupromazine, Levomepromazine), piperidines (Mesoridazine, Thioridazine) and piperazines (Trifluoperazine, Fluphenazine, Prochlorperazine and Perphenazine)

FIG. 7: Dose-dependent effect of phenothiazine derivatives on the dispersion of meningococcal aggregates. Bacteria were grown in suspension for 2 h then Phenothiazine derivatives were added at the indicated concentrations. The effect on bacterial aggregation was observed 30 min after treatment by phase contrasts microscopy.

FIG. 8: Trifluoperazine prevents meningococcal adhesion to endothelial cells.

Nm2C4.3 grown in suspension were pretreated in the absence or in the presence of 30 μM Trifluoperazine for 30 minutes before adhesion to human dermal microvascular endothelial cells (HDMECs) for 30 minutes. Infection was allowed to proceed for further 30, 60 or 90 minutes before fixation and immunostaining using anti-Nm2C4.3 antibody and Alexa Fluor 633 Phalloidin. (A) Representative fluorescence microscopy showing bacterial colony (white dots) formed at the surface of the endothelial cells. (B) Quantification of the bacterial colonization was performed using Image J software.

FIG. 9: Trifluoperazine induces the dispersion of compact meningococcal microcolonies formed at the surface of infected human endothelial cells.

Human bone marrow microvascular endothelial cells (HBMEC) were infected with Nm2C4.3 for 1 h 30, treated with Trifluoperazine (10-40 μM) for 30 minutes, before fixation and immunofluorescence analysis using anti-Nm2C4.3 antibody and Alexa Fluor 633 Phalloidin. (A) Representative fluorescence microscopy showing bacterial colony (white dots) formed at the surface of the endothelial cells. (B) Quantification of the bacterial colonization was performed using Image J software.

FIG. 10: Trifluoperazine exerts a cytoprotective effect on endothelial cells infected by N. meningitidis: Effect on cytoskeleton remodelling and endothelial cell junction integrity.

(A) Experimental procedure: HDMEC were infected for 2 h with Nm2C4.3, treated for 1 h with gentamicin (150 μg/ml) and then 15 min with Trifluoperazine (50 μM) (or solvent alone as a control) and left in medium for 1 h before fixation. (B) Upper panels: Representative fluorescence microscopy showing the effect on the endothelial cell cytoskeleton remodelling analysed by immunostaining of Ezrin and Actin. Lower panels: Effect on the endothelial cell junction integrity was analysed by immunostaining of Actin and VE-cadherin. (C) Quantification of the bacterial colonization (upper panel) Ezrin recruitment and cortical actin polymerization at sites of bacterial adhesion (lower panel) was performed using Image J software.

FIG. 11: Trifluoperazine exerts a cytoprotective effect on endothelial cells infected by N. meningitidis: Trifluoperazine inhibits PECAM-1 recruitment at the bacterial adhesion sites.

(A) Monolayers of HBMECs were non-infected or infected for 2 h with meningococci and the effect on the localisation of the endothelial cell junction proteins PECAM-1 and □-catenin was analysed by immunofluorescence analysis. Arrows point at PECAM-1 molecules recruited at bacterial adhesion sites (B) HBMECs were infected for 2 h with meningococci in the presence or in the absence of 5 μM Trifluoperazine and the effect on junctional PECAM-1 and on the endothelial cell cytoskeleton remodelling (Ezrin, Actin) was analysed by immunofluorescence analysis. (C) Quantification of Actin and PECAM-1 recruitment at sites of bacterial adhesion was performed using Image J software.

FIG. 12: Trifluoperazine exerts a cytoprotective effect on endothelial cells infected by N. meningitidis: Effect on basement membrane remodelling.

(A) Experimental procedure: HBMECs grown on a fluorescent matrix (Gelatin-Fitc) were colonized by N. meningitidis for 6 h. They were then treated with 150 μg/ml gentamicin for 1 h, then 15 min with Trifluoperazine (50 μM) (or solvent alone as control) and incubated for further 15 h in the presence of 150 μg/ml gentamicin. (B) Fluorescence microscopy was used to visualize cells stained with labelled phalloidin and the fluorescent matrix. The presence of dark zones corresponds to zones of matrix degradation. (C) Quantification of the percentage of matrix degradation, using Image J software.

FIG. 13: Trifluoperazine induces the dispersion of compact EPEC microcolonies formed at the surface of human endothelial cells.

Human bone marrow microvascular endothelial cells (HBMEC) were infected with a GFP-expressing mutant Enteropathogenic Escherichia coli, which lacks the ATPase escN (EPEC ΔescN-GFP) for 2 h, then treated with Trifluoperazine (10-50 μM) for 30 minutes, before fixation and immunofluorescence analysis using Alexa Fluor 633 Phalloidin. (A) Representative fluorescence microscopy showing bacterial colony (white dots) formed at the surface of the endothelial cells. (B) Quantification of the bacterial colonization was performed using Image J software.

FIG. 14: Trifluoperazine induces the dispersion of compact meningococcal microcolonies formed at the surface of infected human brain endothelial cells.

A. HBMEC were infected with Nm2C4.3 for 1 h 30, treated with Trifluoperazine (10-40 μM) for 30 minutes, before fixation and immunofluorescence analysis using anti-Nm2C4.3 antibody and Alexa Fluor 633 Phalloidin. B. Quantification of the bacterial colonization was performed using Image J software.

FIG. 15: Trifluoperazine induces the dispersion of compact meningococcal microcolonies formed during in situ infection of human brain vessels

A. Immunofluorescence analysis of CD31 (green), N. meningitidis colonies (red) and nucleus (blue) in the cortical region of serial brain sections from the same donor infected with Nm2C4.3 wild-type strain for 1 h, then treated with Trifluoperazine (40 μM for 30 min) or PBS alone, as a control. Scale bars, 20 μm. Images are representative of 2 different human brain sections. B. Mean±s.e.m. of the vascular colonization index. Analysis of 50 sections per brain section, n=2 sections per condition. ***P<0.001, one-way ANOVA.

### EXAMPLES

### Example 1: Trifluoperazine Exerts a Moderate Bactericidal Effect on Meningococci

### Materials and Methods

Nm2C4.3, a piliated capsulated Opa⁻Opc⁻ variant of the serogroup C meningococcal clinical isolate 8013, was cultured in Dulbecco's Modified Eagle Medium (DMEM) 4.5 g/L-Glutamax media 0.1% BSA during two hours at 37° C. 5% CO2. A bacterial suspension at optical density (OD) 0.1 was then distributed in 24-well plate (1 ml/well). After 1 hour of incubation, Trifluoperazine (Sigma #T8516, stock solution in PBS) was added to obtain final concentrations of 10, 20, 30, 40, and 50 μM. Gentamicin (150 μg/ml in DMEM) and PBS alone were used as controls. After 15 min treatment, serial dilutions were performed in physiological water and 100 μl of each dilution were plated on Petri dishes containing GCB solid media (BD, Difco GC media) containing supplements, incubated overnight and the colony forming units were counted.

### Results

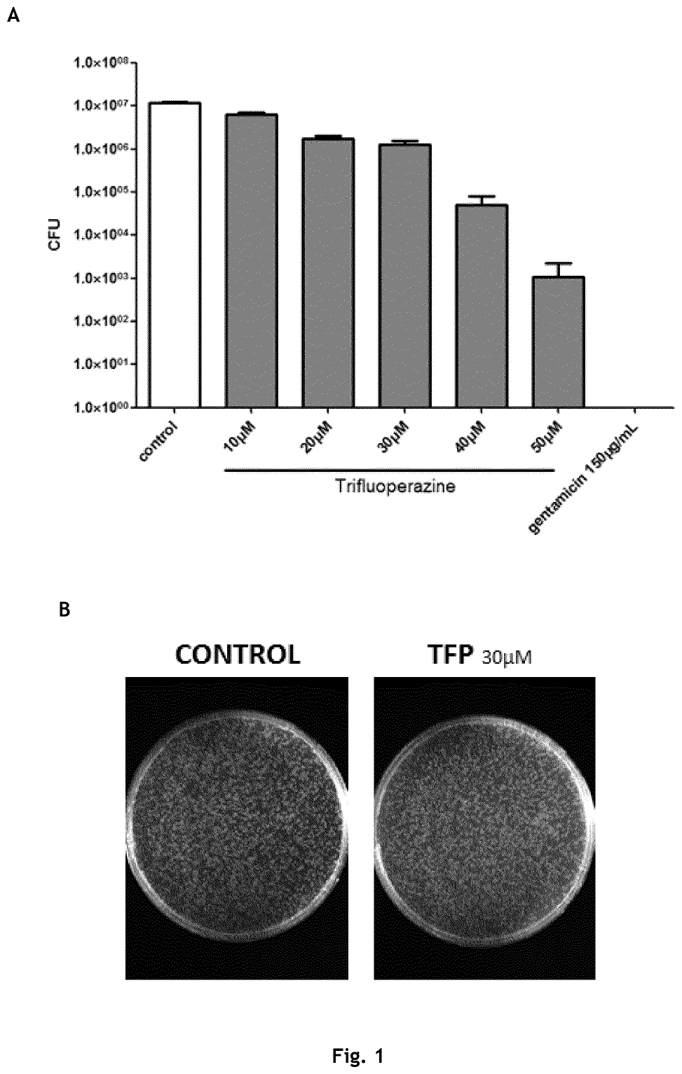

Previous studies showed that Trifluoperazine was a broad-spectrum bactericide for Gram-positive and Gram-negative bacteria, especially active on staphylococci and vibrios (Mazumber et al., 2001). When tested on N. meningitidis, Trifluoperazine also showed some significant antimicrobial activity at concentrations ranging from 10 to 50 μM: the viable count of the culture that contained 10⁷ CFU/ml was reduced to 10⁶ CFU/ml at 10 to 30 μM and dropped to 10³ CFU/ml at 50 μM (FIG. 1). However, this effect was moderate in comparison to antibiotic treatment such as Gentamicin, which killed all bacteria (FIG. 1).

### Conclusions

Trifluoperazine exerts a moderate bactericidal effect on meningococci.

### Example 2: Trifluoperazine Rapidly Induced the Dispersal of Meningococcal Aggregates

### Materials and Methods

N. meninigitidis 2C4.3 strain was grown in suspension in wells of a 24 well plate, containing 1 ml of DMEM medium supplemented with 10% heat-inactivated fetal calf serum. After 2 h of growth, Trifluoperazine (or control vehicle) was added at various concentrations ranging from 10 to 40 μM for 20 min and the bacterial aggregates were visualized over time using a phase-contrast microscope.

### Results

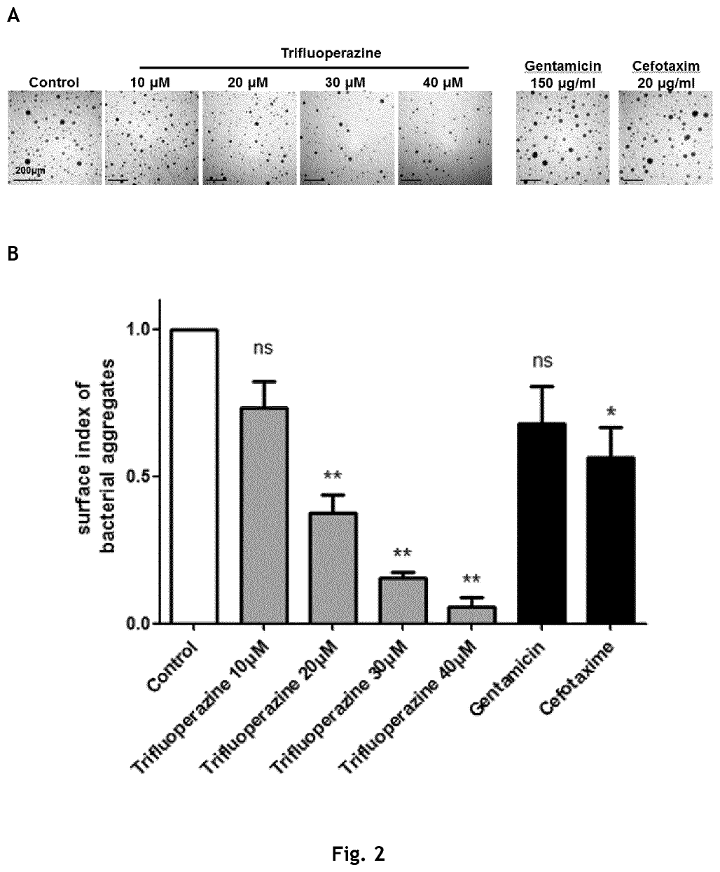

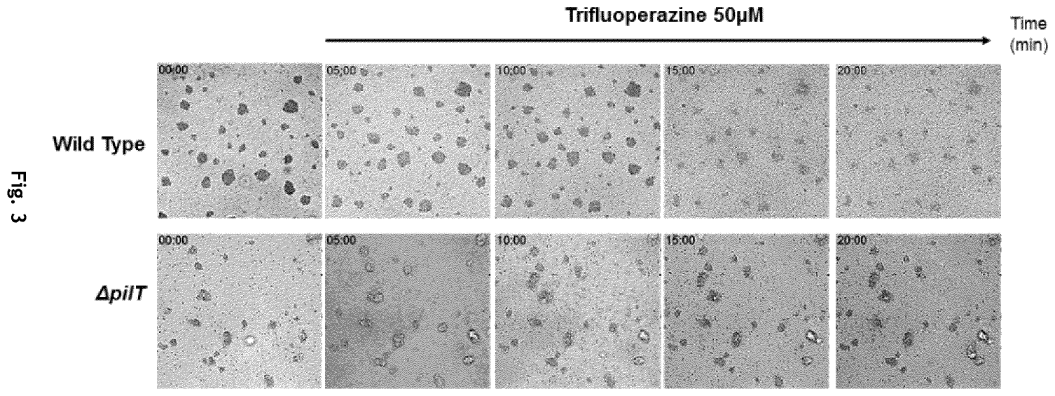

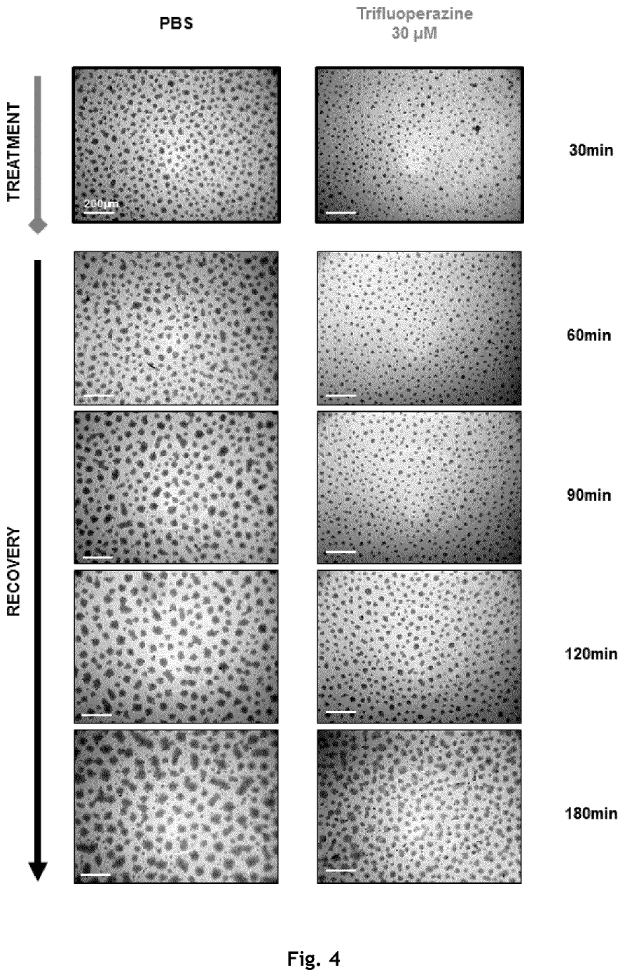

When grown in suspension in liquid culture, meningococci form bacterial aggregates due to interbacterial interactions promoted by their type IV pili (Helaine et al., 2005; Pelicic, 2008). When added to bacterial aggregates, which were pre-formed for two hours, Trifluoperazine induced their dispersion (FIG. 2). This effect was dose-dependent (between 10-40 μM) (FIG. 2), and time laps video microscopy showed that this effect occurs within minutes (10-15 min) after addition of Trifluoperazine (FIG. 3). Upon removal of Trifluoperazine this effect was reversible (FIG. 4). In contrast to Trifluoperazine, addition of conventional antibiotics used in the treatment of meningococcaemia, such as Gentamicin (150 μg/ml) or Cefotaxim (20 μg/ml), did not significantly induce the dispersion of bacterial aggregates formed in suspension.

### Conclusions

In contrast to conventional antibiotics used for the treatment of meningococcemia (Cefotaxim or Gentamicin), Trifluoperazine induced the dispersal of bacterial aggregates formed in suspension.

### Example 3: Trifluoperazine Induces the Loss of Bacterial Type IV Pili

### Materials and Methods

N. meninigitidis 2C4.3 strain and the isogenic derivative mutant PilT, where the pilT gene was interrupted by an erythromycin-resistance cassette (Pujol et al., 1999), were grown in suspension in DMEM medium supplemented with 10% heat-inactivated fetal calf serum. PilT belongs to a highly conserved protein family homologous to AAA-type motor proteins and is proposed to cause the retraction of type IV pili by disassembling the pilin subunits at the base of the fiber (Morand et al., 2004). After 2 h of growth, Trifluoperazine (or control vehicle) was added at 50 μM for 20 min. The effect on bacterial aggregates were visualized over time using a phase-contrast microscope or the bacterial suspensions were fixed in 4% Paraformaldehyde for 10 min, centrifuged at 1000 rpm for 5 minutes and the bacterial pellets washed in PBS. After negative staining with 1% phosphotungstic acid, bacteria were analysed by transmission electron microscopy, using a JEOL 1011 microscope.

### Results

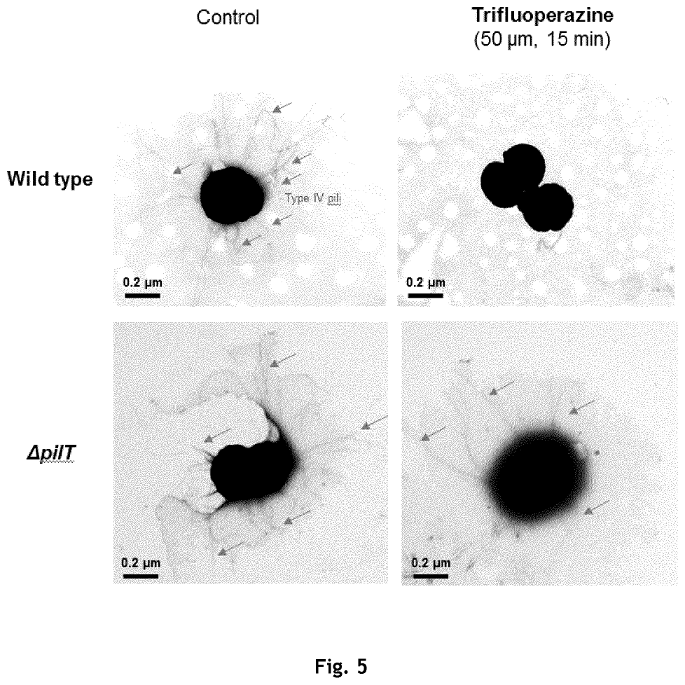

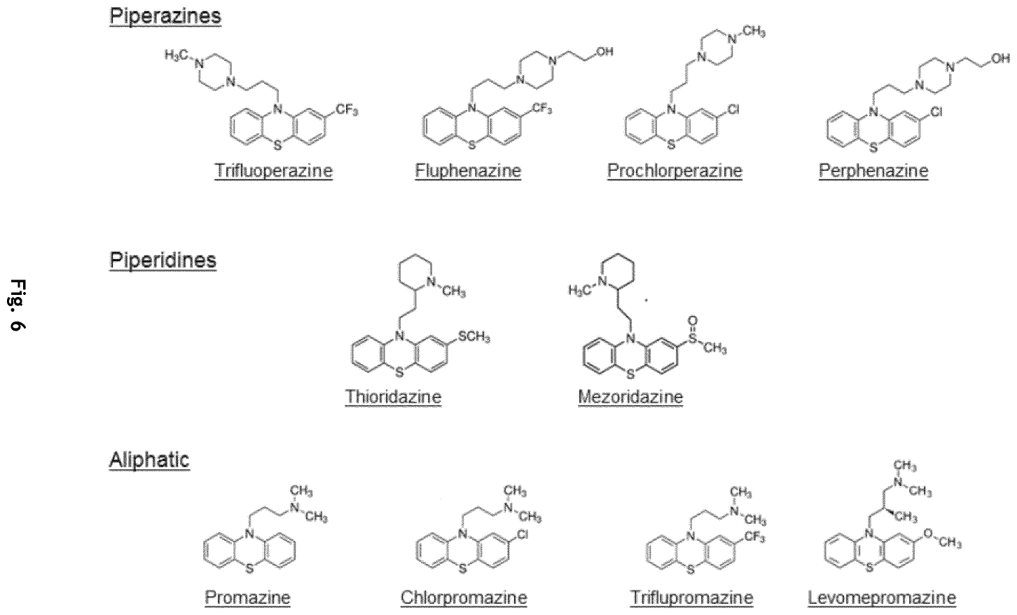

Results showed that Trifluoperazine exerts a rapid effect on meningococcal aggregation, a function carried by the type IV pili. These polymeric pilus fibers are highly dynamic molecular structures that switch between elongation and retraction. The hexameric ATPase PilT is required for type IV pilus retraction. Interestingly, Trifluoperazine treatment had no effect on the aggregation of a derivative PilT mutant strain, unable to retract its type IV pili (FIG. 3). We therefore examined the effect of Trifluoperazine on the piliation status of meningococci by transmission electron microscopy. As shown in FIG. 5, while control 2C4.3 meningococci (treated with solvent alone) express type IV pili on their surface, (i.e. long filamentous appendages assembled in bundles, pointed by arrows), these structures were no longer observed on bacteria treated with 50 μM Trifluoperazine for 30 minutes. In contrast, Trifluoperazine treatment of the PilT mutant did not affect its surface expression of type IV pili (FIG. 5).

### Conclusions

Trifluoperazine induces a drastic loss of the surface expression of meningococcal type IV pili. Trifluoperazine affects the pilus dynamics by exerting a direct or indirect effect on the PilT ATPase, responsible for Type IV pilus retraction.

### Example 4: The Phenothiazine Derivatives Compounds, Piperidines and Piperazines, all Induce the Dispersal of Meningococcal Aggregates

### Materials and Methods

N. meninigitidis 2C4.3 strain was grown in suspension in wells of a 24 well plate, containing 1 ml of DMEM medium supplemented with 10% heat-inactivated fetal calf serum. After 2 h of growth, various concentrations (0.5 to 80 μM) of phenothiazine-derivative compounds or control vehicle (PBS or DMSO) were added to the wells for 30 min and the bacterial aggregates were visualized using a phase-contrast microscope. Were tested phenothiazine-derivative compounds of:
the Piperazine group: Trifluoperazine (Sigma #T8516), Fluphenazine (Sigma #F4765), Prochlorperazine (Sigma #P9178) Perphenazine (Sigma #P6402);
the Piperidine group: Thioridazine (Sigma #T9025) and Mesoridazin (Sigma #M4068);
the Aliphatic group: Chlorpromazine (Sigma #C8138), Promazine (Sigma #P6656), Triflupromazine (Sigma #1686003) and levomepromazine (Sigma #L0500000).

### Results

Trifluoperazine belongs to a large family of phenothiazine derivatives classified into three groups that differ with respect to the substituent on nitrogen: the aliphatic compounds (bearing acyclic groups), the “piperidines” (bearing piperidine-derived groups), and the piperazine (bearing piperazine-derived substituents) (FIG. 6). We addressed here the effect of the other derivatives compounds on the dispersion of meningococcal aggregates.

All piperazine and piperidine compounds tested induced the dispersion of meningococcal aggregates formed in suspension, in a dose-dependent manner and with varying efficiencies:

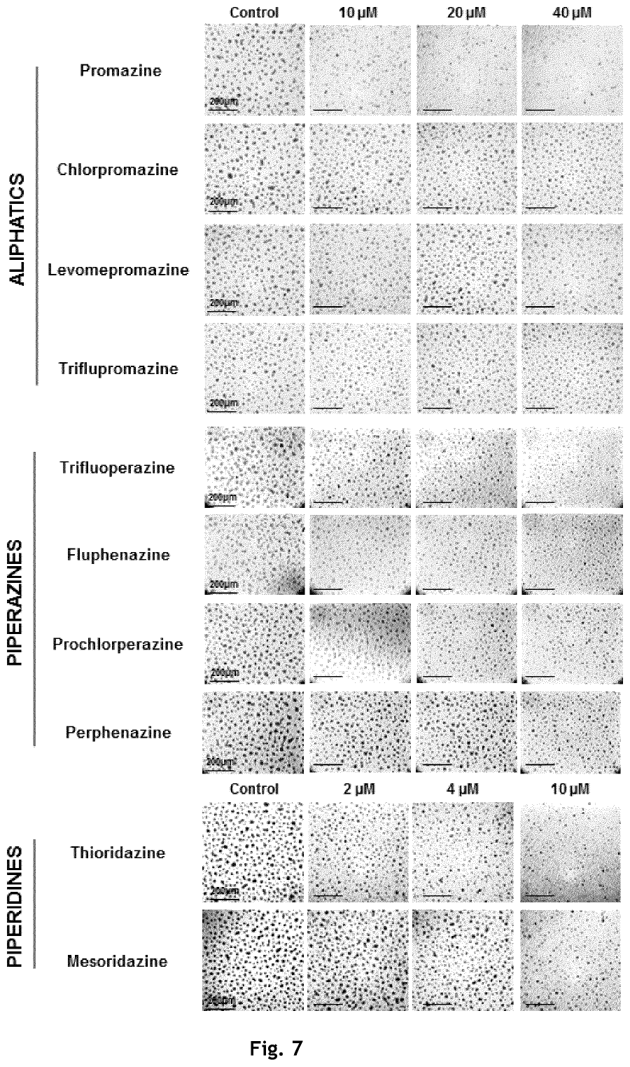

While piperazines induced the dispersal at concentrations ranging from 10 to 50 μM, Piperidines were efficient at 1 to 4 μM (FIG. 7; Table 1).

By contrast, no effects were observed with the aliphatic compounds (Chlorpromazine, Levomepromazine Triflupromazine) in this range of concentrations excepted for Promazine, which inhibited bacterial aggregation with better efficacy than Trifluoperazine (FIG. 7; Table 1).

TABLE 1
Effect of the different phenothiazine-derivative compounds on
meningococcal dispersal.

| | | Concentration promoting |
|---|---|---|
| | | efficient meningococcal |
| Structure | Name | dispersal (~80%) |
| 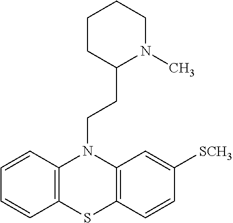 [SMILES: CSc1ccc2c(c1)N(CCC1CCCCN1C)c1ccccc1S2] | Thioridazine | 4 μM |
| 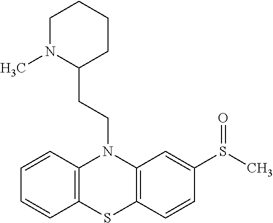 [SMILES: CN1CCCCC1CCN1c2ccccc2Sc2ccc(S(C)=O)cc21] | Mesoridazine | 6 μM |
| 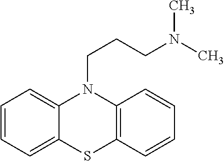 [SMILES: CN(C)CCCN1c2ccccc2Sc2ccccc21] | Promazine | 10 μM |
| 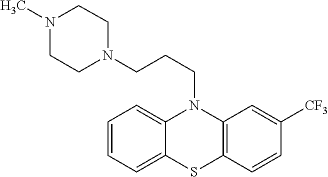 [SMILES: CN1CCN(CCCN2c3ccccc3Sc3ccc(C(F)(F)F)cc32)CC1] | Trifluoperazine | 30 μM |
| 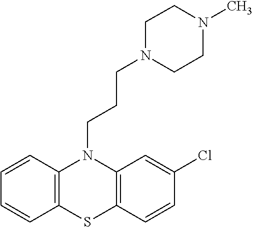 [SMILES: CN1CCN(CCCN2c3ccccc3Sc3ccc(Cl)cc32)CC1] | Prochlorperazine | 30 μM |
| 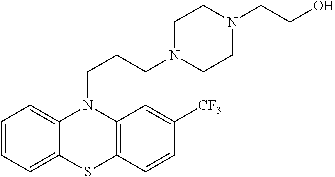 [SMILES: OCCN1CCN(CCCN2c3ccccc3Sc3ccc(C(F)(F)F)cc32)CC1] | Fluphenazine | 40 μM |
| 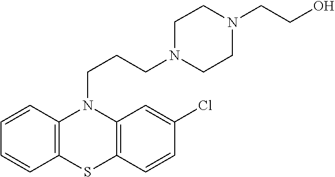 [SMILES: OCCN1CCN(CCCN2c3ccccc3Sc3ccc(Cl)cc32)CC1] | Perphenazine | 40 μM |
| 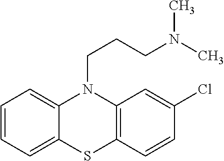 [SMILES: CN(C)CCCN1c2ccccc2Sc2ccc(Cl)cc21] | Chlorpromazine | No effect |
| 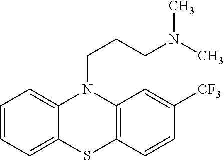 [SMILES: CN(C)CCCN1c2ccccc2Sc2ccc(C(F)(F)F)cc21] | Triflupromazine | No effect |
| 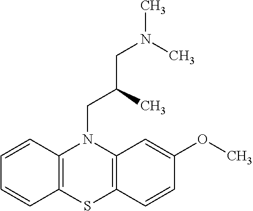 [SMILES: COc1ccc2c(c1)N(C[C@H](C)CN(C)C)c1ccccc1S2] | Levomepromazine | No effect |

### Conclusions

The effect of Trifluoperazine on the dispersion of meningococcal aggregates is common to the tested members of the piperazine and piperidine groups of phenothiazine derivatives.

### Example 5: Trifluoperazine Induces the Dispersion of Compact Meningococcal Microcolonies Formed at the Surface of Infected Human Endothelial Cells

### Materials and Methods

HBMEC, a human endothelial cell line isolated from bone marrow capillaries (Schweitzer et al., 1997) were grown in Dulbecco's Modified Eagle Medium (DMEM) 4.5 g/L-Glutamax (ThermoFischer) 10% FBS. Cells were grown on Thermanox coverslips coated with gelatin 2% (BD Difco #214340) for 2 days to reach confluency. Cells were then infected with a suspension of Nm2C4.3 (˜10⁷ CFU/ml) in DMEM/FBS during 30 minutes to allow bacterial adhesion. Cells were then washed three times with medium to remove non-adherent bacteria and infection was pursued for 1 h 30: adherent bacteria grew at the endothelial cell surface and formed micro-colonies. Trifluoperazine was then added to obtain final concentrations of 10, 20, 30 and 40 μM. After incubation for 30 minutes, cells were washed and fixed in 4% Paraformaldehyde for 10 min, washed three times with PBS, and permeabilized with 0.1% Triton X-100 in PBS for 10 min. Cells were blocked for 30 min with 3% BSA in PBS and were incubated for 2 h with the primary antibodies (Rabbit polyclonal anti-Nm2C4.3 strain, kindly provided by Dr X. Nassif, INEM, Paris). After three washes with PBS, cells were incubated for 1 h with Alexa Fluor 491-conjugated anti-rabbit IgG (Jackson ImmunoResearch Laboratories), together with Alexa Fluor 633 Phalloidin (Thermofischer) and DAPI (0.5 mg ml-1, Sigma Aldrich) to stain Actin and nuclei, respectively. Labelled preparations were mounted in Glycergel media (DAKO) and image acquisitions were performed with a DM16000 microscope (Leica, X40). Quantification was done with ImageJ software (NIH). Results are presented as a vascular colonisation index corresponding to the area occupied by the fluorescently labelled bacteria per fields in relation to the area occupied by the fluorescently labelled human endothelial cells (Actin staining). Statistical analysis were performed with Student t test.

### Results

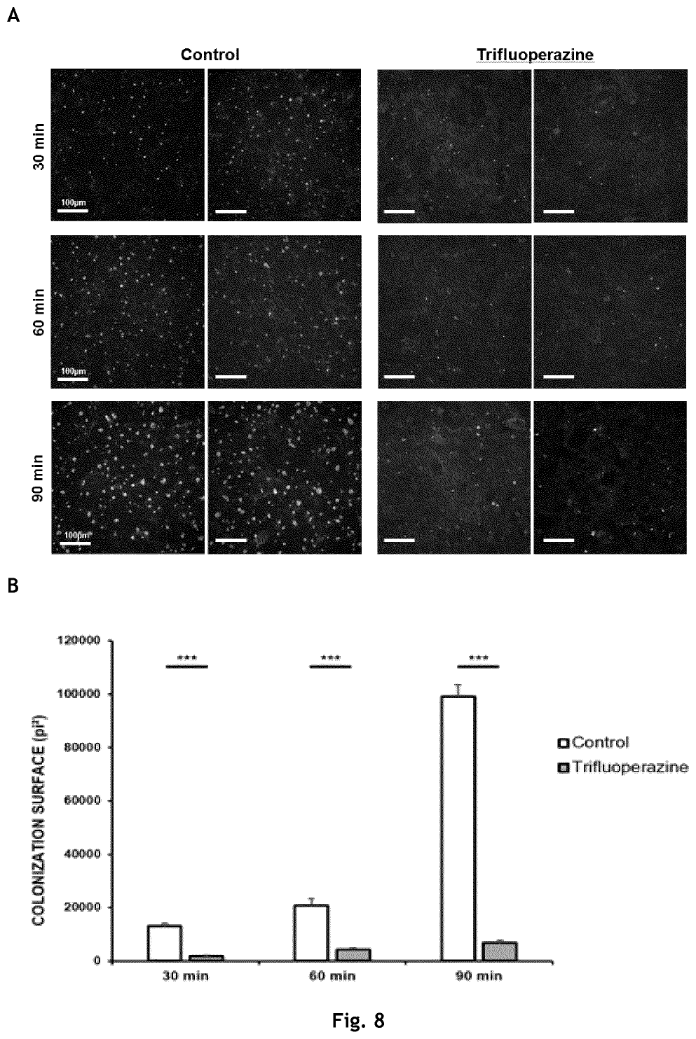

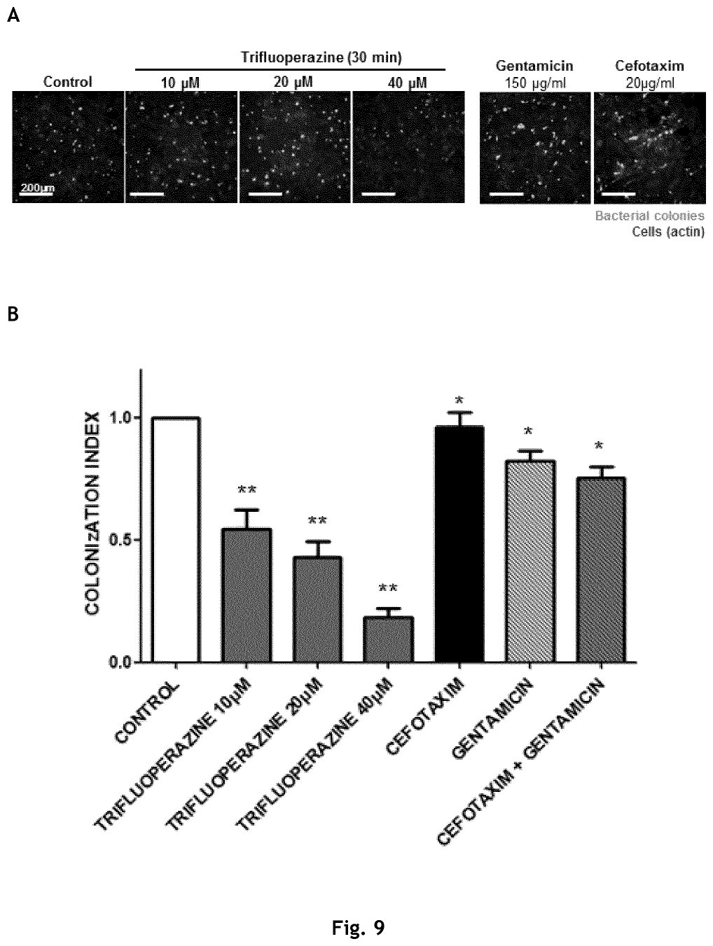

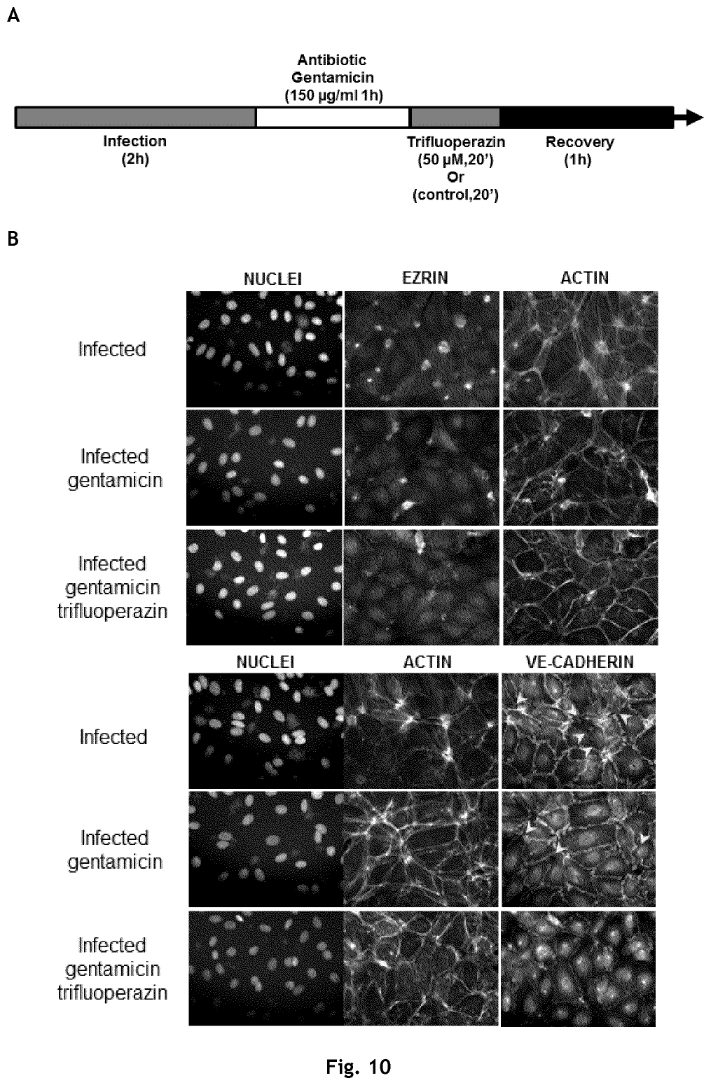

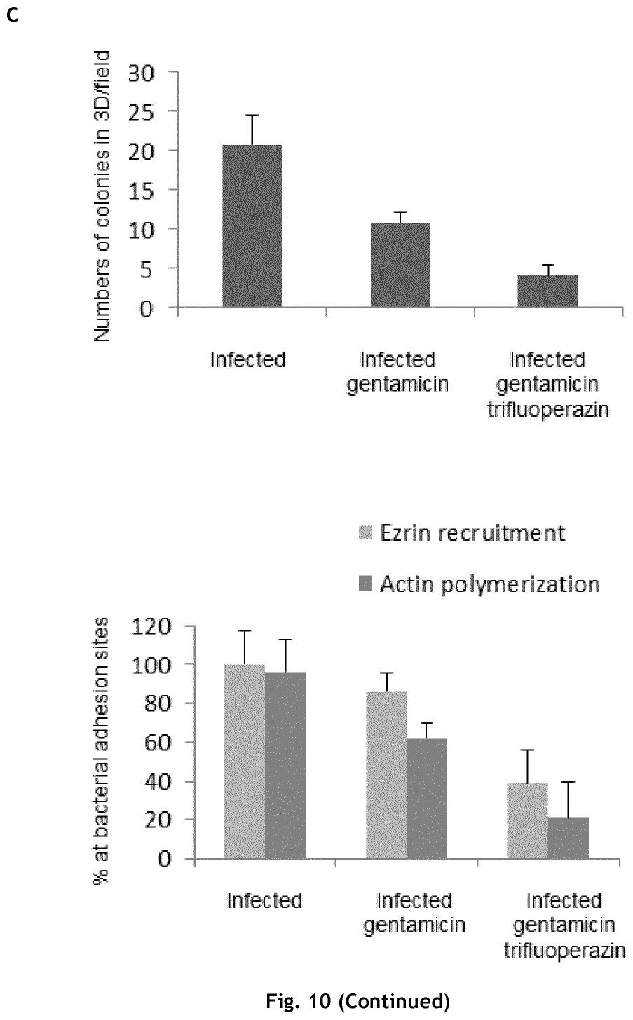

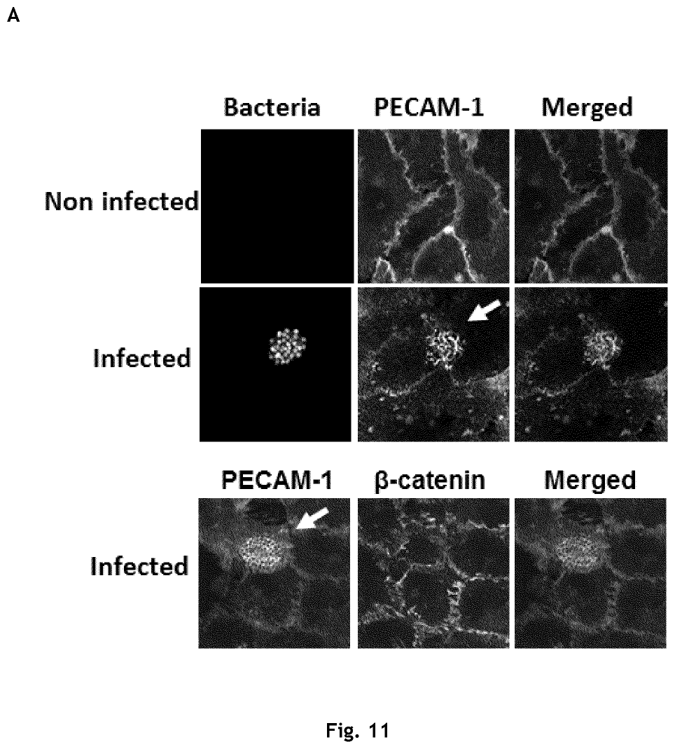

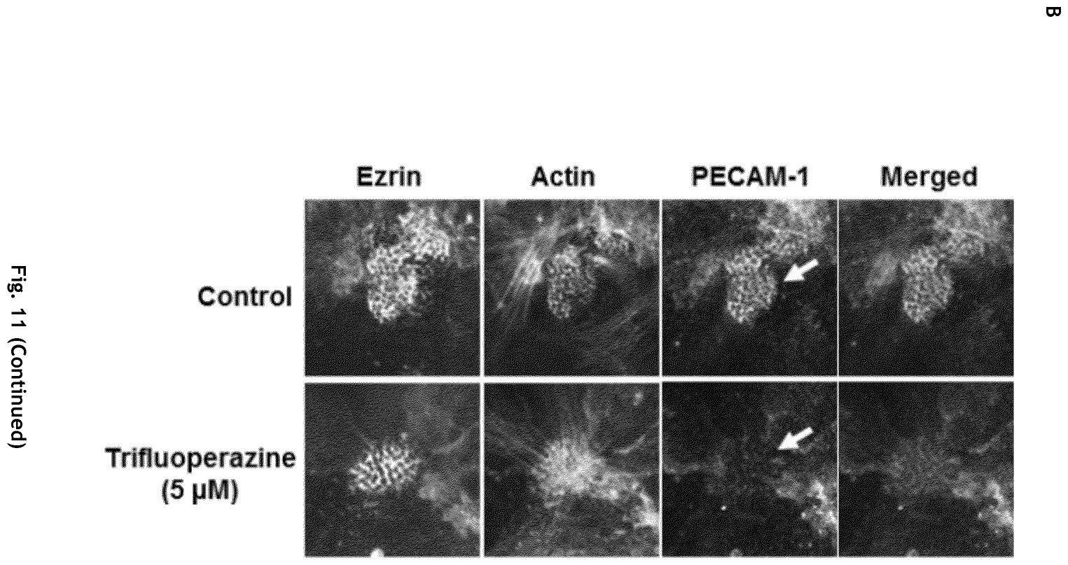

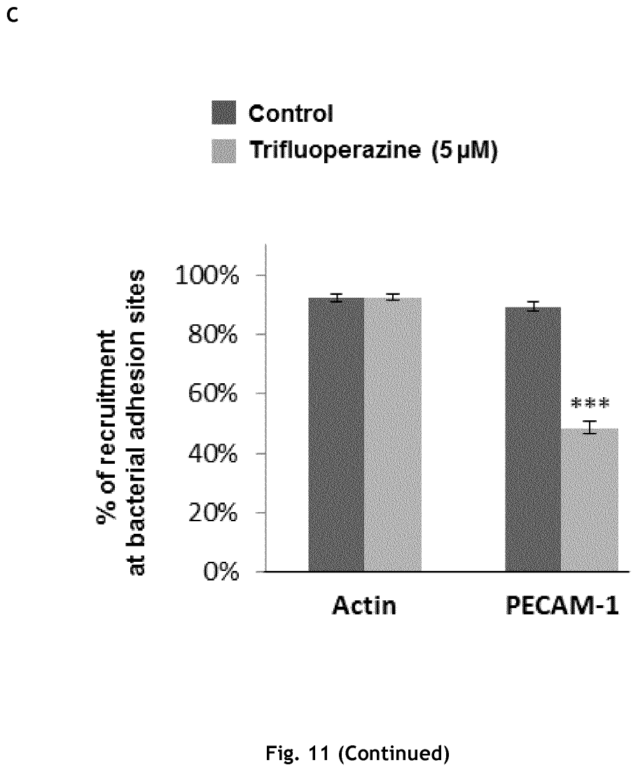

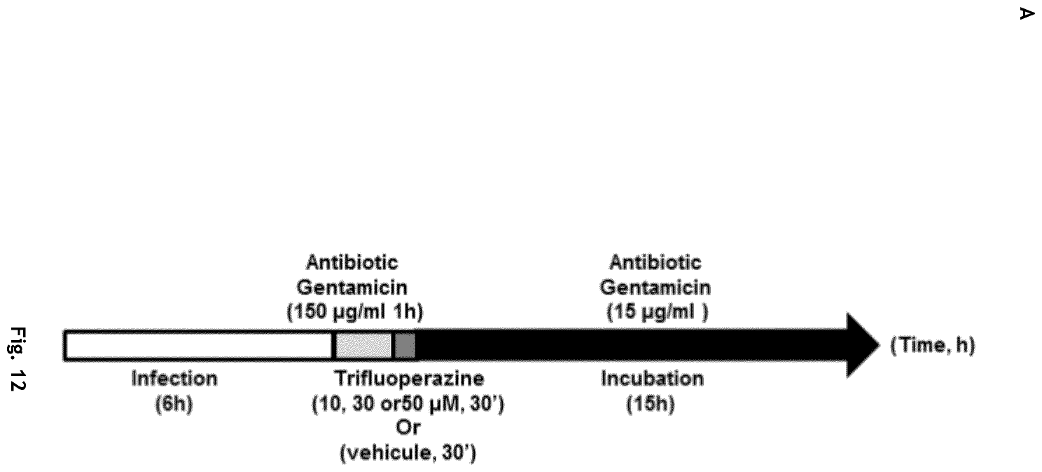

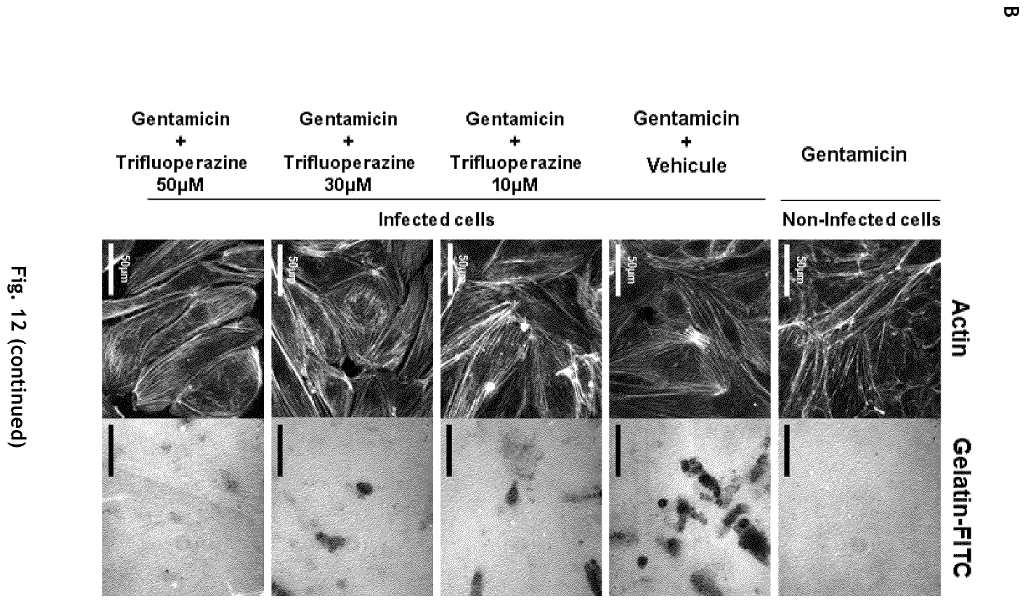

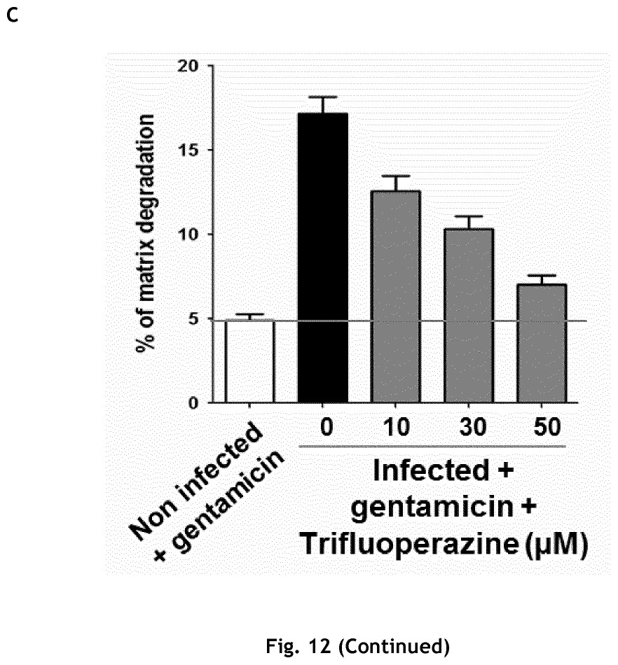

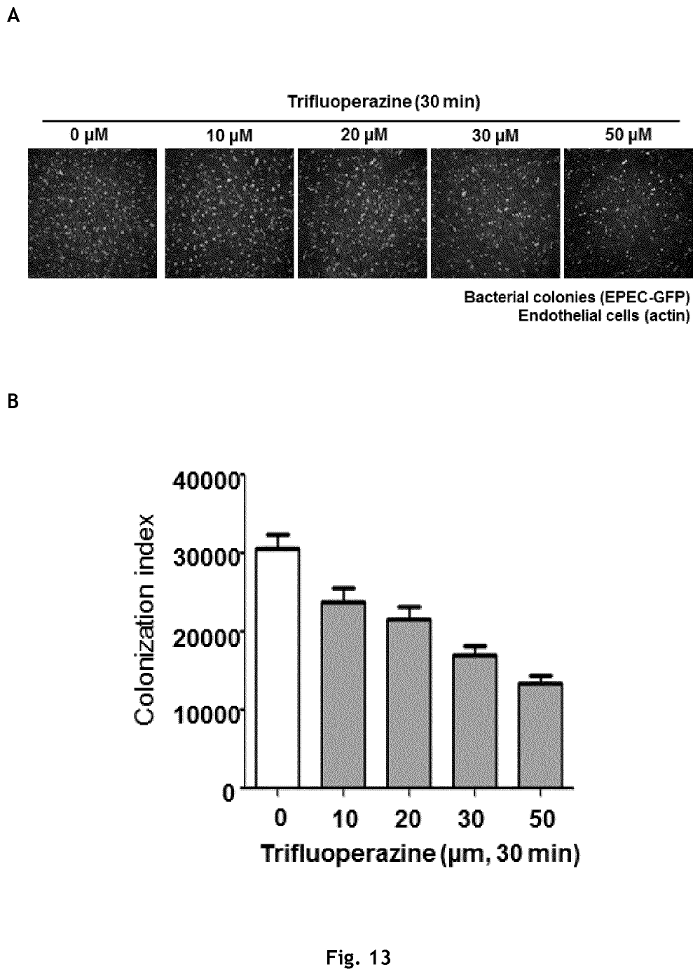

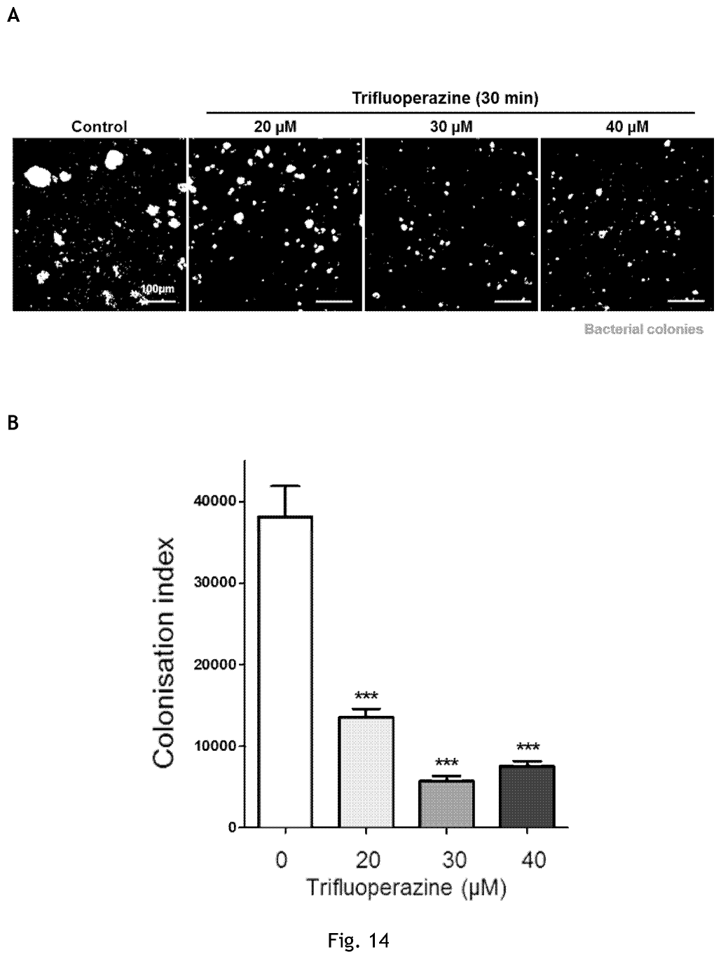

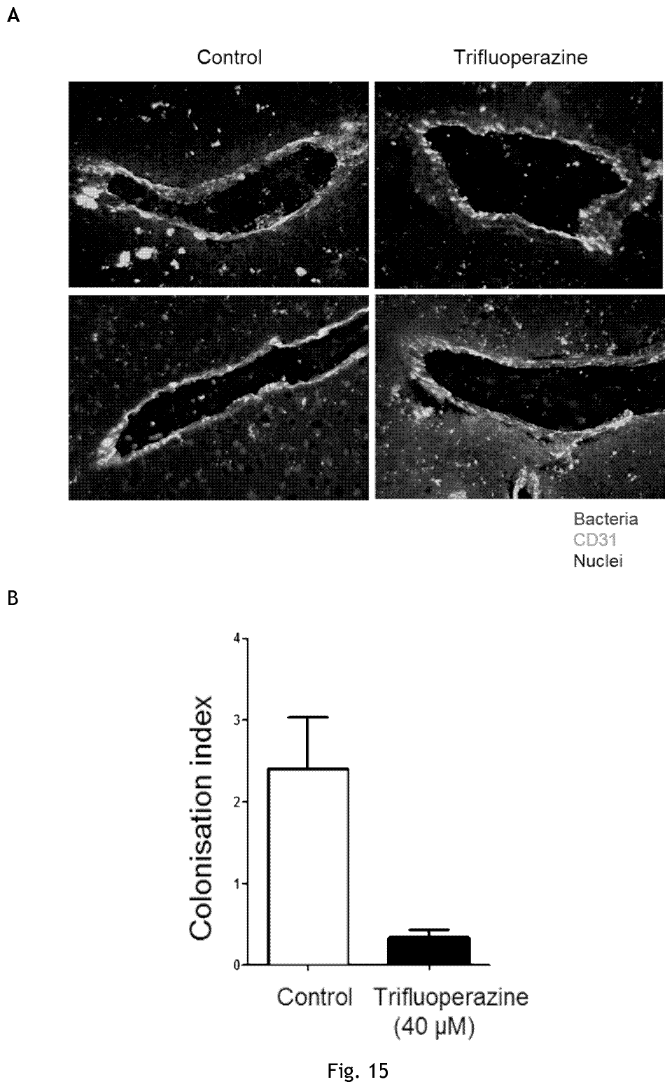

After their initial adhesion on human endothelial cells, meningococci rapidly proliferate at the endothelial cell surface and form compact microcolonies, a process referred to as vascular colonization (Melican and Dumenil, 2012). This intimate interaction of meningococci with endothelial cells leads to deregulated inflammatory and coagulation processes, endothelial dysfunction and, ultimately, the breach of endothelial barriers and bacterial dissemination into perivascular tissues (Coureuil et al., 2014; Join-Lambert et al., 2013). As expected, when pre-treated with 30 μM Trifluoperazine bacteria did no longer adhere to and/or form bacterial colony at the endothelial cells surface (FIG. 8). More interestingly, when Trifluoperazine was applied to compact meningococcal microcolonies already established at the endothelial cell surface, treatment for 30 min induced their dispersion. This effect was dose-dependent (between 10-40 μM) and observed on both a human bone marrow endothelial cell line (FIG. 9) and on primary human endothelial cells isolated from dermal microvessels (FIG. 10). In contrast to Trifluoperazine, addition of conventional antibiotics used in the treatment of meningococcaemia, such as Gentamicin (150 μg/ml) or Cefotaxim (20 μg/ml), poorly induced the dispersion of these microcolonies.

### Conclusions

In contrast to conventional antibiotics used for the treatment of meningococcemia (Cefotaxim or Gentamicin), Trifluoperazine induces the dispersal of bacterial microcolonies that form at the endothelial cell surface.

### Example 6: Trifluoperazine Exerts a Cytoprotective Effect on Endothelial Cells Infected by N. meningitidis: Effect on Cytoskeleton Remodelling and Endothelial Cell Junction Integrity

### Materials and Methods

HDMECs were grown in their specific culture medium (Promocell #C-12210) and confluent monolayers were infected with 2C4.3. Briefly, bacteria were precultured in prewarmed cell culture medium for 1 h 30 min at 37° C. 5% CO₂. The OD₆₀₀ was adjusted to 0.1 and HDMECs were then overlaid with bacteria for 30 min (MOI of 100). Unbound bacteria were removed by three washes in cell culture media and infection was allowed to proceed for 1 h at 37° C. 5% CO₂. Gentamicin was added at 150 μg/ml for 1 h, then after three washes in cell culture media, Trifluoperazine was applied where mentioned at 50 μM for 20 min. After three washes in cell culture media, cells were allowed to recover for 1 h in cell culture media. At the indicated time, cells were fixed with 4% Paraformaldehyde for 10 min. Immunolabelling was then performed as above described in example 4, using DAPI, Alexa Fluor 633 Phalloidin, Polyclonal antisera raised against Ezrin, obtained from Pr P. Mangeat (CRBM, Montpellier, France) and anti-human VE-Cadherin (eBioscience #BMS158).

When indicated, HBMECs were grown on Thermanox coverslips coated with gelatin 2% for 2 days to reach confluency. Cells were then infected as above described in the presence or in the absence of 5 μM Trifluoperazine for 2 h. Cells were washed and fixed in 4% paraformaldehyde and immunolabelling was performed with a polyclonal antisera raised anti-Nm2C4.3 strain, anti-human PECAM-1 mouse monoclonal, clone HEC7 (ABCAM ab119339) or rabbit polyclonal 177 raised against PECAM-1, obtained from WA Muller (Northwestern University, Chicago, Ill., US), anti-0 catenin (05-482, UBI), polyclonal antisera raised against Ezrin, Alexa Fluor 633 Phalloidin. Secondary antibodies were from Jackson ImmunoResearch Laboratories. Image acquisitions were performed on a Leica DM16000 with Yokogawa CSU-X1M1 system and CoolSnap HQ2 (Photometrics). Quantifications were done using ImageJ software (NIH).

### Results

After adhesion, N. meningitidis promotes host cell signalling events, involving Ezrin, Src and Cortactin as main organizers of actin polymerization and receptor clustering (Eugene et al., 2002; Hoffmann et al., 2001; Lambotin et al., 2005; Merz et al., 1999; Soyer et al., 2014). These events promote formation of membrane protrusions that surround bacteria and increase the membrane surface to which the bacteria adhere. This step is critical to resist the shear stress conditions that prevail in vivo (Mikaty et al., 2009). Furthermore, bacteria promote signalling events leading to the delocalization of cell-cell junction molecules such as VE-cadherin, ZO-1 or Claudin-5, at the sites of bacterial adhesion where these proteins are sequestered (Coureuil et al., 2010; Coureuil et al., 2009). VE-cadherin is an endothelial specific cell-cell adhesion molecule that plays a pivotal role in the formation, maturation and remodelling of the vascular wall. These events result in the destabilization of the endothelial cell-cell junctions, increased permeability and bacterial diffusion within surrounding tissues (Coureuil et al., 2014; Dupin et al., 2012). These bacteria-to-cell signaling events are dependent on the interaction between Type IV and endothelial cell receptors (Bernard et al., 2014; Coureuil et al., 2010). We therefore addressed whether Trifluoperazine, by inducing the loss of meningocococcal Type IV pili and the subsequent dispersion of the bacteria that colonize the endothelial cell surface, would prevent the subsequent vascular damages promoted by bacteria-induced signalling events.

Indeed, in control infected conditions, meningococcal microcolonies formed at the endothelial cell surface induced a strong recruitment of Ezrin, and an important cortical actin polymerization at the bacterial adhesion sites, accompanied by the loss of the continuous staining of VE-cadherin at the endothelial cell junctions and the formation of gaps between cells (pointed by arrows, FIG. 10). Upon treatment with antibiotics (Gentamicin 150 μg/ml for 1 h), although it reduced the number of colonies present at the cell surface, residual colonies (still induced Ezrin recruitment, cortical actin polymerization at bacterial adhesion sites, accompanied by the discontinuous distribution of VE-cadherin and the occurrence of gaps between the endothelial cells (FIG. 10), indicating that antibiotic-treated bacteria still promoted signalling events leading to vascular alterations. When used in combination with gentamicin, Trifluoperazine, induced the dispersion of most of the bacterial colonies at the endothelial cell surface. Few Ezrin recruitment was observed under residual bacteria, F-actin was polymerized at the periphery of the cells, accompanied with a continuous staining of VE-cadherin at the intercellular junctions and the absence of gap between cells, therefore indicating that the clearance of bacteria promoted by Trifluoperazine preserved endothelial cell junction integrity (FIG. 10).

Interestingly, we also observed that upon adhesion to endothelial cells, meningococci promote the massive delocalization of PECAM-1/CD31 from the intercellular junctions of the endothelial cells to the bacterial adhesion sites (FIG. 11A). Treatment with low concentrations of Trifluoperazine (5 μM) did not affect bacterial colonization, nor prevented Ezrin recruitment or Actin polymerization at bacterial sites; however, it prevented PECAM-1 recruitment at bacterial adhesion sites (FIGS. 11B, 11C). These results indicate that, besides its effect on bacterial clearance obtained at concentrations ranged from 10 to 50 μM that preserved endothelial cell junction integrity, Trifluoperazine may further improve vascular protection at lower concentrations by acting directly on infected endothelial cells (i.e. by inhibiting the massive PECAM-1 delocalization from the endothelial cell junctions).

### Conclusions

In contrast to conventional antibiotics used for the treatment of meningococcemia, Trifluoperazine can stop the endothelial cells from receiving intracellular signals that results in their large scale systemic dysregulation. These compounds exert a vasculoprotective effect on infected endothelial cells by acting both on bacteria and on infected cells.

### Example 7: Trifluoperazine Exerts a Cytoprotective Effect on Endothelial Cells Infected by N. meningitidis: Effect on Basement Membrane Remodelling

### Materials and Methods

Plastic coverslips (13 mm diameter) (Nalgen #174950) were washed in ethanol 70%, dried then coated with poly-L-lysine 1 mg/ml for 20 min at room temperature. After one wash in sterile PBS, 0.5% Glutaraldehyde was added for 15 min at room temperature. After three washes in PBS, Gelatin-FITC (0.2 mg/ml Invitrogen #G13187) was added for 10 minutes at room temperature in the dark. Coverslip were then washed with sterile PBS and treated with 5 mg/ml sodium borohydride for 3 min. After three washes in sterile PBS, 10⁵ HDMECs (PromoCell #C-12210) were seeded per well in their PromoCell culture medium and incubated overnight at 37° C. 5% CO₂. The day after, cells were infected with N. meningitidis Nm2C4.3, as above described. Infection was allowed to proceed for 5 h 30 min before Gentamicin treatment at 150 mg/ml for 1 h. After three washes in cell culture medium, Trifluoperazine was added for 20 min, washed with the culture medium and were incubated overnight in cell culture media containing 15 mg/ml Gentamicin. Cells were then fixed in 4% Paraformaldehyde for 10 min, stained with Phalloidin 633 for 1 hour and mounted in glycergel (DAKO). Image acquisitions were performed on a Leica DMI6000 with Yokogawa CSU-X1M1 system and CoolSnap HQ2 (Photometrics). Quantifications were performed using ImageJ software (NIH).

### Results

To visualize a potential protective effect of Trifluoperazine on the basement membrane integrity, HDMECs were grown on a fluorescent gelatin matrix (Gelatin-FITC), the appearance of dark areas corresponding to the zones of matrix degradation (FIG. 12). In the absence of infection, a homogenous fluorescence was observed, showing the integrity of this basement membrane, only few zones of degradation were observed (<5% of the basement membrane). Because it takes 16 to 24 h to observe matrix degradation, the time that metalloproteinases involved are synthesized and degrade the matrix, antibiotics treatment was applied after few hours of infection to avoid bacterial overload and subsequent cell death, due to culture medium consumption. Despite antibiotic treatment with 150 μg/ml Gentamicin, infection of endothelial cells was accompanied by a degradation of about 20% of the basement membrane 24 h post-infection. Treatment with Trifluoperazine reduced this drastic degradation of the matrix in a dose-dependent manner (by 30 to 60%) (FIG. 12).

### Conclusions

All these results demonstrate that treatment with Trifluoperazine induces a cyprotective effect on endothelial cells, by preserving the integrity of their basement membrane.

### Example 8: Trifluoperazine Induces the Dispersion of Compact Microcolonies Formed by Enteropathogenic Escherichia coli at the Surface of Infected Human Endothelial Cells

### Materials and Methods

HBMEC were grown on Thermanox coverslips coated with gelatin 2% for 2 days to reach confluency. Cells were then infected for 1 h with a GFP-expressing mutant Enteropathogenic Escherichia coli (EPEC), which lacks the ATPase escN. This strain is unable to translocate effector proteins and is more prone to form pilus-dependent microcolony at the host cell surface (Jensen et al., 2015). Cells were then washed three times with medium to remove non-adherent bacteria and infection was pursued for 1 extra hour, to allow bacterial growth at the endothelial cell surface to form microcolonies. Trifluoperazine was then added to obtain final concentrations of 10, 20, 30 and 50 μM. After incubation for 30 minutes, cells were washed and fixed in 4% Paraformaldehyde for 10 min, washed three times with PBS. Cells were incubated for 1 h with Alexa Fluor 633 Phalloidin (Thermofischer) together with DAPI (0.5 mg ml-1, Sigma Aldrich) to stain Actin and nuclei, respectively. Labelled preparations were mounted in Glycergel media (DAKO) and image acquisitions were performed with a DM16000 microscope (Leica, X20). Quantification was done with ImageJ software (NIH). Results are presented as a vascular colonization index corresponding to the area occupied by the fluorescently labelled bacteria per fields in relation to the area occupied.

### Results

To address whether trifluoperazine would affect vascular colonization by other piliated pathogens, we used Enteropathogenic Escherichia coli (EPEC), as, similarly to N. meningitidis, EPEC requires Type IV bundle-forming pili to autoaggregate and to form microcolonies on human cells (Bieber et al., 1998; Moreira et al., 2006). Type IV pili are also essential for EPEC virulence, as EPEC mutants hindered for microcolony formation have been shown to be highly attenuated for virulence in human volunteers (Bieber et al., 1998).

Human endothelial cells were infected for 1 h with a GFP-expressing mutant Enteropathogenic Escherichia coli (EPEC), which lacks the ATPase escN, as this strain, unable to translocate effector proteins and to induce pedestal formation, is more prone to form pilus-dependent microcolony at the host cell surface (Jensen et al., 2015).

After their initial adhesion on human endothelial cells, ΔescN EPEC-GFP rapidly proliferated at the endothelial cell surface and formed compact microcolonies. Treatment for 30 min with Trifluoperazine (10 to 50 μM) induced the dispersion of these microbial colonies in a dose-dependent manner (FIG. 13).

### Conclusions

The effect of Trifluoperazine on the dispersal of bacterial microcolonies is not limited to N. meningitidis but also apply to other bacterial pathogens that require type IV pili to colonize human cells.

### Example 9: Trifluoperazine Induces the Dispersion of Compact Microcolonies Established at the Surface of Human Brain Endothelial Cells

### Materials and Methods

HCMEC/D3, a well-established human brain endothelial cell line (Weksler et al, 2013) was grown in EBM-2 basal medium (Lonza, Walkersville, Md., USA) supplemented with 5% Fetal Bovine Serum “Gold”, 10 mM HEPES (PM Laboratories GmbH, Pasching, Austria), 1% Penicillin-Streptomycin, 1% chemically defined lipid concentrate (Invitrogen Ltd, Paisley, UK), 1.4 μM hydrocortisone, 5 μg·ml⁻¹ ascorbic acid and 1 ng·ml⁻¹ bFGF (Sigma-Aldrich, St. Louis, Mo.). Cells were grown on Thermanox coverslips coated with rat collagen I for 4 days at 37° C. in a humidified incubator in 5% CO₂. Cells were then infected with a suspension of Nm2C4.3 (˜10⁷ CFU/ml) in EBM2/FBS during 30 minutes to allow bacterial adhesion. Cells were then washed three times with medium to remove non-adherent bacteria and infection was pursued for 1 h 30: adherent bacteria grew at the endothelial cell surface and formed micro-colonies. Trifluoperazine was then added to obtain final concentrations of 10, 20, 30 and 40 μM. After incubation for 30 minutes, cells were washed and fixed in 4% Paraformaldehyde for 10 min, washed three times with PBS, and permeabilized with 0.1% Triton X-100 in PBS for 10 min. Cells were blocked for 30 min with 3% BSA in PBS and were incubated for 2 h with the primary antibodies (Rabbit polyclonal anti-Nm2C4.3 strain, kindly provided by Dr X. Nassif, INEM, Paris). After three washes with PBS, cells were incubated for 1 h with Alexa Fluor 491-conjugated anti-rabbit IgG (Jackson ImmunoResearch Laboratories), together with Alexa Fluor 633 Phalloidin (Thermofischer) and DAPI (0.5 mg ml-1, Sigma Aldrich) to stain Actin and nuclei, respectively. Labelled preparations were mounted in Glycergel media (DAKO) and image acquisitions were performed with a DM16000 microscope (Leica, X40). Quantification was done with ImageJ software (NIH). Results are presented as a vascular colonisation index corresponding to the area occupied by the fluorescently labelled bacteria per fields in relation to the area occupied by the fluorescently labelled human endothelial cells (Actin staining). Statistical analysis were performed with Student t test.

### Results

Trifluoperazine was applied to compact meningococcal microcolonies already established at the surface of human brain endothelial cells, treatment for 30 min induced their dispersion. This effect was dose-dependent (between 10-40 μM) (FIG. 14).

### Conclusion

Trifluoperazine induces the dispersal of bacterial microcolonies that form at the surface of human brain endothelial cells.

### Example 10: Effect of Trifluoperazine on the Colonization of Human Brain Vessels, Using an In Situ Meningococcal Infection Model of Fresh Human Frontal Brain Tissues Obtained from Deceased Normal Subjects

### Materials and Methods

In situ infection of fresh human brain sections was performed as described previously (Bernard et al, 2014). Fresh human brain sections were obtained from frontal lobe specimens of macroscopically and histologically normal brain (confirmed by a neuropathologist) of individuals referred to the Department of Forensic Medicine for unexplained out-of-hospital sudden death (consent forms ML1094, PFS 10-008, ClinicalTrials.gov NCT00320099 from The Institutional Review Boards of the Poincaré Hospital, Versailles-Saint Quentin University and the French “Agence de la Biomédecine”). After freezing of the brain tissue with isopentane cooled in liquid nitrogen, the sections, 7 μm thick, containing leptomeninges, cortical ribbon and the underlying white matter were immobilised on Superfrost™ plus microscope slides and stored at −80° C. Defrosted sections were rehydrated in PBS for 5 min and incubated for 1 h with medium containing 0.1% BSA prior to infection with suspensions of bacteria (2×10⁷ bacteria in 150 μl of medium containing 0.1% BSA) for 1 h at 37° C. Sections were then treated for 30 min Trrifluoperazine 40 μM or PBS alone as a control and were then gently washed horizontally 5 times and fixed in PAF 4% for 10 min at RT. Adherent meningococci were detected by immunofluorescence analysis: brain sections were incubated with the following primary antibodies for 2 h in PBS/BSA 0.1%: monoclonal anti-human CD31/PECAM-1 mouse monoclonal antibody (clone HEC7, ABCAM, ab119339) and a rabbit polyclonal serum anti-Nm 2C4.3 strain (1:3000). Alexa-conjugated phalloidin and DAPI (0.5 mg/ml) were added to Alexa-conjugated secondary antibodies for 1 h. After additional washing, coverslips were mounted in glycergel (Dako). Entire samples were scanned using a Lamina (Perkin Helmer) and were further analysed using confocal microscopy (spinning disk Leica, 63x). Quantification analysis of the images (n=50 per section) was performed using ImageJ software (NIH). Results are presented as a vascular colonisation index corresponding to the area occupied by the fluorescently labelled bacteria in relation to the human vessel area delineated by the anti-PECAM-1 staining, from 2 independent sections per condition.

### Results

The effect of trifluoperazine on the colonization of human brain vessels were further analyzed using an in situ meningococcal infection model of fresh human frontal brain tissues obtained from deceased normal subjects, as described previously (Bernard et al, 2014). In this setting, histological and anatomical characteristics of the brain vessels are conserved. Meningococci incubated with tissue sections established specific tight association with brain vessels, reminiscent of neuropathological findings in patients with meningococcal meningitis and adhesion relied on the expression of type IV pili (Bernard et al, 2014).

Upon infection, meningococci developed microcolonies immediately adjacent to CD31-positive endothelial cells (FIG. 15. A.). Consistent with in vitro cellular models, treatment of infected brain sections with Trifloperazine 40 μM for 30 min induced the dispersal of these meningococcal microcolonies (FIG. 15. A et B.), reducing by 80% the vascular colonization of the human brain vessels.

Therefore, trifluoperazine reduced in situ infection of human brain vessels, indicating that it might reduce the signs of meningitis.

### Conclusion

In this study, trifluoperazine and related phenothiazines were identified to blocked all the functions carried by the type IV pili (bacterial competence, twitching motility, aggregation and adhesion to inert surface or host endothelial cells) in different bacterial pathogens.

Trifluoperazine has shown to induce within minutes the retraction of the meningococcal Type IV pili. In contrast to conventional antibiotics used for the treatment of meningococcemia (Cefotaxim or Gentamicin), trifluoperazine promotes the dispersal of compact microcolonies already formed at the surface of peripheral and brain endothelial cells in vitro and reduce subsequent endothelial alteration. Trifluoperazine induces the dispersal of compact microcolonies formed in situ in a meningococcal infection model of human frontal brain tissues. Finally, when used in vivo, in mice engrafted with human skin, trifluoperazine prevents the massive colonization of the human dermal vasculature, reduces the signs of intravascular coagulation, reduces incidence of vascular alteration. Importantly, while cefotaxime treatment increased by 2 fold this inflammatory response, most likely by promoting the release of various Pathogen-associated molecular patterns that activate innate immune response, trifluoperazine alone or in combination with cefotaxime, drastically reduced the hallmark of vascular inflammation. By inducing bacterial clearance, trifluoperazine can prevent an overwhelming inflammatory response, therefore conferring a potential advantage over antibiotics treatment.

These findings reveal an unexpected outcome of phenothiazine therapy for the targeting of a major bacterial virulence factor that could be developed to prevent and treat bacterial diseases, in particular, as antibiotic adjuvant therapy for treatment of meningococcal diseases.

### BIBLIOGRAPHIC REFERENCES

Bernard, S C et al. Pathogenic utilizes CD147 for vascular colonization. Nat Med. 7:725-31 (2014).
Bieber, D., S. W. Ramer, C. Y. Wu, W. J. Murray, T. Tobe, R. Fernandez, and G. K. Schoolnik. 1998. Type IV pili, transient bacterial aggregates, and virulence of enteropathogenic Escherichia coli. Science. 280:2114-2118.
Brandtzaeg, P. [symbol] van Deuren, M. Classification and pathogenesis of meningococcal infections. Methods Mol. Biol. 799, 21-35 (2012).
Brown, D. R. et al. Systematic functional analysis reveals that a set of seven genes is involved in fine-tuning of the multiple functions mediated by type IV pili in Neisseria meningitidis. Infect. Immun. 78, 3053-3063 (2010).
Carrithers, M., S. Tandon, S. Canosa, M. Michaud, D. Graesser, and J. A. Madri. 2005. Enhanced susceptibility to endotoxic shock and impaired STAT3 signaling in CD31-deficient mice. Am J Pathol. 166:185-196.
Craig, L. [symbol] Li, J. Type IV pili: paradoxes in form and function. Curr. Opin. Struct. Biol. 18, 267-277 (2008).
Coureuil, M., S. Bourdoulous, S. Marullo, and X. Nassif. 2014. Invasive meningococcal disease: a disease of the endothelial cells. Trends Mol Med. 20:571-578.
Coureuil, M., H. Lecuyer, M. G. Scott, C. Boularan, H. Enslen, M. Soyer, G. Mikaty, S. Bourdoulous, X. Nassif, and S. Marullo. 2010. N. meningitidis Hijacks a beta2-adrenoceptor/beta-Arrestin pathway to cross brain microvasculature endothelium. Cell. 143:1149-1160.
Coureuil, M., G. Mikaty, F. Miller, H. Lecuyer, C. Bernard, S. Bourdoulous, G. Dumenil, R. M. Mege, B. B. Weksler, I. A. Romero, P. O. Couraud, and X. Nassif. 2009. Meningococcal type IV pili recruit the polarity complex to cross the brain endothelium. Science. 325:83-87.
Doulet, N. et al. Neisseria meningitidis infection of human endothelial cells interferes with leukocyte transmigration by preventing the formation of endothelial docking structures. J. Cell Biol. 173, 627-637 (2006).
Dupin, N., H. Lecuyer, A. Carlotti, C. Poyart, M. Coureuil, J. Chanal, A. Schmitt, M. C. Vacher-Lavenu, M. K. Taha, X. Nassif, and P. C. Morand. 2012. Chronic meningococcemia cutaneous lesions involve meningococcal perivascular invasion through the remodeling of endothelial barriers. Clin Infect Dis. 54:1162-1165.
Eugene, E., I. Hoffmann, C. Pujol, P. O. Couraud, S. Bourdoulous, and X. Nassif. 2002. Microvilli-like structures are associated with the internalization of virulent capsulated N. meningitidis into vascular endothelial cells. J Cell Sci. 115:1231-1241.
Fernandez-Martin, L., B. Marcos-Ramiro, C. L. Bigallera, M. Graupera, R. J. Cain, N. Reglero-Real, A. Jimenez, E. Cernuda-Morollon, I. Correas, S. Cox, A. J. Ridley, J. Millan. 2012. Crosstalk between reticular adherens junctions and platelet endothelial cell adhesion molecule-1 regulates endothelial barrier function. Artherioscler Thromb Vasc Biol. 32:e90-102.
Ferrero, E., M. E. Ferrero, R. Pardi, and M. R. Zocchi. 1995. The platelet endothelial cell adhesion molecule-1 (PECAM1) contributes to endothelial barrier function. FEBS Lett. 374:323-326.
Graesser, D., A. Solowiej, M. Bruckner, E. Osterweil, A. Juedes, S. Davis, N. H. Ruddle, B. Engelhardt, and J. A. Madri. 2002. Altered vascular permeability and early onset of experimental autoimmune encephalomyelitis in PECAM-1-deficient mice. J Clin Invest. 109:383-392.
Helaine, S., E. Carbonnelle, L. Prouvensier, J. L. Beretti, X. Nassif, and V. Pelicic. 2005. PilX, a pilus-associated protein essential for bacterial aggregation, is a key to pilus-facilitated attachment of N. meningitidis to human cells. Mol Microbiol. 55:65-77.
Hoffmann, I., E. Eugene, X. Nassif, P. O. Couraud, and S. Bourdoulous. 2001. Activation of ErbB2 receptor tyrosine kinase supports invasion of endothelial cells by N. meningitidis. J Cell Biol. 155:133-143.
Jensen, H. H., H. N. Pedersen, E. Stenkjaer, G. A. Pedersen, F. H. Login, and L. N. Nejsum. 2015. Tir Is Essential for the Recruitment of Tks5 to Enteropathogenic Escherichia coli Pedestals. PLoS One. 10:e0141871.
Join-Lambert, O., H. Lecuyer, F. Miller, L. Lelievre, A. Jamet, L. Furio, A. Schmitt, P. Pelissier, S. Fraitag, M. Coureuil, and X. Nassif. 2013. Meningococcal interaction to microvasculature triggers the tissular lesions of purpura fulminans. J Infect Dis.
Lambotin, M., I. Hoffmann, M. Laran-chich, X. Nassif, P. O. Couraud, and S. Bourdoulous. 2005. Invasion of endothelial cells by N. meningitidis requires cortactin recruitment by a PI3-Kinase/Rac1 signalling pathway triggered by the lipo-oligosaccharide. J Cell Sci. 118:3805-3816.
Lemichez, E., Lecuit, M., Nassif, X. [symbol] Bourdoulous, S. Breaking the wall: targeting of the endothelium by pathogenic bacteria. Nat. Rev. Microbiol. 8, 93-104 (2010).
Lerolle, N., Carlotti, A., Melican, K., Aubey, F., Pierrot, F., Diehl, J. L., Caille, V., Hékimian, G., Gandrille, S., Mandet, C., Bruneval, P., Dumenil, G., and Borgel, D. 2013. Assessment of the interplay between blood and skin vascular abnormalities in adult purpura fulminans. Am J Respir Crit Care Med. 188:684-692.
Maas, M., M. Stapleton, C. Bergom, D. L. Mattson, D. K. Newman, and P. J. Newman. 2005. Endothelial cell PECAM-1 confers protection against endotoxic shock. Am J Physiol Heart Circ Physiol. 288:H159-164.
Mazumder, R., K. Ganguly, S. G. Dastidar, and A. N. Chakrabarty. 2001. Trifluoperazine: a broad spectrum bactericide especially active on staphylococci and vibrios. Int J Antimicrob Agents. 18:403-406.
Melican, K., and G. Dumenil. 2012. Vascular colonization by N. meningitidis. Curr Opin Microbiol. 15:50-56. Merz, A. J., C. A. Enns, and M. So. 1999. Type IV pili of pathogenic Neisseriae elicit cortical plaque formation in epithelial cells. Mol Microbiol. 32:1316-1332.
Mikaty, G., M. Soyer, E. Mairey, N. Henry, D. Dyer, K. T. Forest, P. Morand, S. Guadagnini, M. C. Prevost, X. Nassif, and G. Dumenil. 2009. Extracellular bacterial pathogen induces host cell surface reorganization to resist shear stress. PLoS Pathog. 5:e1000314.
Morand, P. C., E. Bille, S. Morelle, E. Eugene, J. L. Beretti, M. Wolfgang, T. F. Meyer, M. Koomey, and X. Nassif. 2004. Type IV pilus retraction in pathogenic Neisseria is regulated by the PilC proteins. Embo J. 23:2009-2017.
Moreira, C. G., K. Palmer, M. Whiteley, M. P. Sircili, L. R. Trabulsi, A. F. Castro, and V. Sperandio. 2006. Bundle-forming pili and EspA are involved in biofilm formation by enteropathogenic Escherichia coli. J Bacteriol. 188:3952-3961.
Nassif, X. et al. Roles of pilin and PilC in adhesion of Neisseria meningitidis Neisseria meningitidis to human epithelial and endothelial cells. Proc. Natl. Acad. Sci. USA 91, 3769-3773 (1994).
Roland Nau and Helmut Eiffert. Modulation of Release of Proinflammatory Bacterial Compounds by Antibacterials: Potential Impact on Course of Inflammation and Outcome in Sepsis and Meningitis. Clin Microbiol Rev. 2002 January; 15(1): 95-110.
Park, S. Y., T. A. DiMaio, E. A. Scheef, C. M. sorenson, N. Sheibani. 2010. PECAM-1 regulates proangiogenic properties of endothelial cells through modulation of cell-cell and cell-matrix interactions. Am J Physiol Cell Physiol. 299:1468-1484.
Pelicic, V. 2008. Type IV pili: e pluribus unum? Mol Microbiol. 68:827-837.
Pujol, C., E. Eugene, M. Marceau, and X. Nassif. 1999. The meningococcal PilT protein is required for induction of intimate attachment to epithelial cells following pilus-mediated adhesion. Proc Natl Acad Sci USA. 96:4017-4022.
Schweitzer, K. M., P. Vicart, C. Delouis, D. Paulin, A. M. Drager, M. M. Langenhuijsen, and B. B. Weksler. 1997. Characterization of a newly established human bone marrow endothelial cell line: distinct adhesive properties for hematopoietic progenitors compared with human umbilical vein endothelial cells. Lab Invest. 76:25-36.
Soyer, M., A. Charles-Orszag, T. Lagache, S. Machata, A. F. Imhaus, A. Dumont, C. Millien, J. C. Olivo-Marin, and G. Dumenil. 2014. Early sequence of events triggered by the interaction of N. meningitidis with endothelial cells. Cell Microbiol. 16:878-895.
Stephens, D. S., Greenwood, B. [symbol] Brandtzaeg, P. Epidemic meningitis, meningococcaemia, and Neisseria meningitidis. Lancet 369, 2196-2210 (2007).
Stephens, D. S., Whitney, A. M., Rothbard, J. [symbol] Schoolnik, G. K. Pili of Neisseria meningitidis. Analysis of structure and investigation of structural and antigenic relationships to gonococcal pili. J. Exp. Med. 161, 1539-1553 (1985).
Virji, M. Pathogenic neisseriae: surface modulation, pathogenesis and infection control. Nat. Rev. Microbiol. 7, 274-286 (2009).
Virji, M. et al. Pilus-facilitated adherence of Neisseria meningitidis to human epithelial and endothelial cells: modulation of adherence phenotype occurs concurrently with changes in primary amino acid sequence and the glycosylation status of pilin. Mol. Microbiol. 10, 1013-1028 (1993).
Weksler, B. et al. The hCMEC/D3 cell line as a model of the human blood brain barrier. Fluids Barriers CNS. 10, 16 (2013).
Winther-Larsen, H. C. et al. Neisseria gonorrhoeae PilV, a type IV pilus-associated protein essential to human epithelial cell adherence. Proc. Natl. Acad. Sci. USA 98, 15276-15281 (2001).
Wong, M. A, S. N. Harbour, J. L. Wee, L. M. Lau, R. K. Andrews, D. E. Jackson. 2004. Proteolytic cleavage of platelet endothelial cell adhesion molecule-1 (PECAM-1/CD31) is regulated by a calmodulin-binding motif. FEBS Letter. 568:70-78.
Yazdankhah, S. P. [symbol] Caugant, D. A. Neisseria meningitidis: an overview of the carriage state. J. Med. Microbiol. 53, 821-832 (2004).

1. A method for treating and/or preventing meningitis caused by bacteria carrying Type IV pili in patients diagnosed with or at risk of developing such infection, comprising the step of administering a therapeutically effective amount of a phenothiazine derivative or a pharmaceutical salt thereof, wherein said phenothiazine derivative is a compound of formula (I): 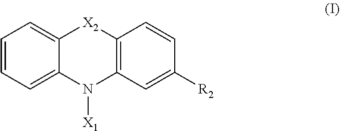
X₁ is a C₁-C₆ alkyl substituted by R₁;
wherein R₁ is YR₃R₄, wherein
Y is C or N,
If Y is N then R₃ and R₄ are independently of each other H, (C₁-C₆)alkyl,
or R3 and R4 form together with Y, a ring selected from an heteroaryl or an heterocyclyl group, said ring being optionally substituted by one or more groups selected from:
(C₁-C₆)alkyl optionally substituted by OH or O(C═O)(C1-C10)alkyl; or
an amide group, and
If Y is C then R₃ and R₄ form together with Y, a ring selected from an heteroaryl or an heterocyclyl group, said ring being optionally substituted by (C₁-C₆)alkyl groups optionally substituted by OH;
R₂ is H, an halogen (preferably Cl or F), CF₃, a (C₁-C₆)alkoxy, S(O)(C₁-C₆)alkyl, SO₂ (C₁-C₆) alkyl, SO₃H, CN, a (C₁-C₆)alkyl, a (C₁-C₆)thioalkoxy, NO₂
X₂ is S or SO₂.

2. The method of claim 1, wherein said phenothiazine derivative is selected from the group consisting of promazine, thioridazine, mesoridazine, trifluoperazine, prochlorperazine, fluphenazine, and perphenazine.

3. The method of claim 1, wherein said phenothiazine derivative is thioridazine, mesoridazine, or trifluoperazine.

4. The method of claim 1, wherein said bacteria carrying Type IV pili are selected from the group consisting of Streptococcus pneumoniae, Neisseria meningitidis, Haemophilus sp., notably Haemophilus influenzae, and Listeria monocytogenes, and is preferentially Neisseria meningitidis.

5. The method of claim 1, wherein vascular damages are prevented.

6. The method of claim 1, wherein circulatory collapse is prevented.

7. The method of claim 1, wherein said phenothiazine derivative is administered intravenously or intra-muscularly.

8. The method of claim 1, comprising the further administration of an antibiotic.

9. The method of claim 1, wherein said antibiotic is selected from the group consisting of beta-lactams and aminoglycosides, and/or dexamethasone.

10. The method of claim 9, wherein said antibiotic is a beta-lactam, preferably a cephalosporin of third generation.

11. The method of claim 8, wherein said phenothiazine derivative and said antibiotic are administered simultaneously, sequentially, or separately.

12. A method for treating and/or preventing meningitis caused by bacteria carrying Type IV pili in patients diagnosed or at risk of developing such infection, comprising the step of administering a pharmaceutical composition comprising a phenothiazine derivative and an antibiotic selected in the group consisting of beta-lactams and aminoglycosides, and/or dexamethasone.

13. The method according to claim 12, wherein meningitis is caused by bacteria carrying Type IV pili selected from the group consisting of Streptococcus pneumonia, Neisseria meningitidis, Haemophilus sp., notably Haemophilus influenza, and Listeria monocytogenes, and is preferably Neisseria meningitidis.

14. A kit comprising a phenothiazine derivative and an antibiotic selected in the group consisting of beta-lactams and aminoglycosides, and/or dexamethasone.
